(19) United States
(12) Patent Application Publication
(10) Pub. No.: US 2006/0292558 A1
(43) Pub. Date: Dec. 28, 2006

(54) Methods and apparatus for protein assay diagnostics

(75) Inventor: Roger O'Neill, Palo Alto, CA (US)

Correspondence Address: Roger O'Neill;Cell Biosciences, 1050 Page Mill Road, Palo Alto, CA, 94303-0803, US

(73) Assignee: Cell Biosciences Inc.

(21) Appl. No.: 11/431,272

(22) Filed: May 9, 2006

Related U.S. Application Data
(63) Continuation-in-part of application No. 11/185,247, filed on Jul. 19, 2005.
(60) Provisional application No. 60/589,139, filed on Jul. 19, 2004.
(60) Provisional application No. 60/617,362, filed on Oct. 8, 2004.

(51) Int. Cl. C12Q 1/70; G01N 33/554; G01N 33/569

(52) U.S. Cl. 435/5; 435/7.32; 435/7.92

(57) ABSTRACT

Automated protein assay apparatus and methods for measuring antibodies against an analyte are described. A standard mixture of one or more analytes is loaded into a capillary, the analytes are resolved by isoelectric focusing and immobilized in the capillary. Serum from a human or non-human subject under analysis is flowed through the capillary and antibodies specific for the immobilized analytes bind to the analytes. A secondary antibody including a detectable marker is introduced, binding to the immobilized antibody-analyte complexes. The locations of the antibody-analyte complexes are detected by means of the detectable markers, revealing the presence of analyte-specific antibodies in the serum.

### METHODS AND APPARATUS FOR PROTEIN ASSAY DIAGNOSTICS

This application is a continuation-in-part of U.S. patent application Ser. No. 11/185,247, filed Jul. 19, 2005 and entitled “METHODS AND DEVICES FOR ANALYTE DETECTION”, which claims benefit under 35 U.S.C. §119(e) to application Ser. No. 60/589,139, entitled “Continuous Determination of Cellular Contents by Chemiluminescence,” filed Jul. 19, 2004 and application Ser. No. 60/617,362, entitled “Determination of Captured Cellular Contents,” filed Oct. 8, 2004, the disclosures of which are incorporated herein by reference in their entireties.

This application relates to methods and apparatus for conducting diagnostic procedures by protein assays.

There are numerous diagnostic tests which are only needed occasionally or that require specialized equipment, complex methods, special training, or variations of all of these considerations. These tests are generally conducted by reference labs, which are large testing facilities that specialize in high volume and specialty testing. Physicians and hospitals use reference labs by sending a lab a patient specimen upon which a diagnostic test is to be performed, generally a tissue sample such as a throat culture or a biopsy, or a bodily fluid such as blood or urine. Since specimens for testing are sent in, a reference lab can serve a large region or even an entire country by the use of air shipments of materials. This ability to provide tests to a larger region enables tests conducted infrequently by one clinic or hospital to be aggregated at one place and conducted economically and efficiently by the reference lab. For instance, a modern automated blood analyzer can test upwards of 120 samples per hour. This aggregation of specialized tests makes the purchase of complex and expensive equipment, as well as the specialized training needed to conduct specialized testing efficiently and precisely, economically justifiable by the reference lab. The concentration of sophisticated equipment and highly trained personnel at the reference lab also facilitates the conduct of tests which can be very significant in the lives of patients, such as tests for hepatitis, cancers, and the human immunodeficiency virus (HIV).

In some cases the testing performed by the reference lab is a two-step process. The first step is a screening step which analyzes a sample for indication of the presence of a target substance or disease state. If the result of the screening step is positive, indicating the presence of the target or state, a definitive test is conducted to precisely and confidently identify the target or disease markers. Some of the diseases which may be tested in this way are Lyme disease, bovine spongiform encephalopathy or BSE, commonly referred to as “mad cow disease,” and HIV.

A typical two-step process for these diseases will begin with a screening test called an enzyme-linked immunosorbent assay (ELISA) followed by a definitive test called a Western blot. In the ELISA test, proteins indicative of a disease such as HIV are evaluated in a serum mixture. The definitive Western blot test then evaluates the components of the patient's serum in a dissociated state by separation and individual identification. The ELISA test is fairly qualitative and used initially to provide a simple positive or negative indication result for a selected pathogen and acts by detecting the presence of an antibody or antigen in the sample. It is often preferred for its ability to estimate ng/ml to pg/ml ordered material in a sample such as a serum, urine or culture supernatant and can be used to screen for past or present infections. An ELISA assay uses two antibodies, one specific to the antigen targeted and another coupled to an enzyme. This second antibody gives the assay its enzyme-linked name and will cause a chromogenic or fluorogenic substrate to produce a detectable signal. Since the ELISA test can be used for evaluation of either the presence of an antigen or the presence of an antibody in a sample, it is useful for determining serum antibody concentrations indicative of HIV or West Nile virus infection for example, and for detecting the presence of antigens indicative of a disease state.

Several variations of ELISA testing may be employed, including indirect ELISA, sandwich ELISA, and competitive binding. An indirect ELISA used for HIV testing, sometimes called an HIV enzyme immunoassay (EIA), may be conducted as follows. Partially purified, inactivated HIV antigen is applied to the well of a microtiter plate. The standard microtiter plate consists of an 8 by 12 array of 96 wells, each about 1 cm deep by 0.7 cm in diameter. The antigen is immobilized by coating it onto the surface of the well. The well is then exposed to patient serum which may contain antibodies to HIV. The patient serum is usually diluted in non-human serum to prevent non-specific antibodies in the serum from binding to the antigen. The antibodies will bind to HIV antigens in the well. The plate is washed so that non-antigen-binding antibodies are washed free of the plate. After the wash only the antibody-antigen complexes remain attached to the well. The second antibody, a conjugate of anti-human immunoglobulin coupled to a substrate-modifying enzyme, is added to the well, which will bind to the antigen-antibody complexes. The plate is washed again so that excess unbound second antibodies are removed. A chromogenic or fluorogenic substrate is applied to the well, which is converted by the enzyme to produce a chromogenic or fluorescent signal. The enzyme acts as an amplifier in the process. Even if only a few enzyme-linked antibodies are present, the enzyme molecules will produce many signal molecules. The signal is detected by a spectrophotometer or other optical device, recorded and analyzed. If the patient's serum contains no antibodies to HIV, no binding to the HIV antigens will occur and consequently the secondary antibodies will not bind, no enzymes will be present to act on the substrate, and no signal will be produced, a negative indication. However if antibodies to HIV are present, binding will occur in both stages of the process and a coloration or optical signal will be produced. The differentiation between a positive or negative result of a chromogenic assay may be done statistically by a trained expert. Several multiples of the standard deviation is often used to differentiate between positive and negative samples. With a fluorogenic assay the optical density of emitted signals may be evaluated to produce a more quantitative result.

It is possible in many instances that false positive or negative results may be obtained. The statistical nature of result interpretation can lead to some ambiguity in the analysis. In some cases women who have experienced pregnancies may possess antibodies directed against human leukocyte antigens (HLA) which are present on host cells used to propagate HIV. These antibodies may result in signal causing a false positive outcome. False negatives can arise if testing occurs in the interval between infection and antibody response in the patient's body. For these reasons a more definitive test, a Western blot, is needed following an HIV ELISA.

In the so-called “sandwich” variation of ELISA, the process starts with a known quantity of antibody bound to the microtiter well, to which an antigen-containing sample is applied. The signal at the end of the process then indicates whether the sample contains the target antigen. In the “competitive binding” variation, unlabeled antibody is incubated in the presence of its antigen and the resulting bound antibody/antigen complexes are added to the antigen-containing sample in the well. This produces an inverse result: the greater the original antigen concentration, the weaker the detectable signal.

In the case of Lyme disease the spirochete Borrelia burgdorferi organism which causes the disease is cultured and applied to the microtiter well. The organism is then incubated with the patient's serum that may contain antibodies directed against the disease. A fluorescent-tagged antiglobulin is added to link with the antibodies present, the plate washed and examined in ultraviolet light. Any antibody to Lyme disease will be attached to the fluorescent antiglobulin and be visible in the ultraviolet light, indicating the presence of the disease. A positive outcome to this screening test is then followed by a definitive Western blot.

The definitive test for these and other diseases is the Western blot, which detects proteins in a given sample of tissue homogenate, serum, or other cellular material. This assay uses gel electrophoresis to separate denatured proteins by mass. The separated proteins are then stabilized in position by transfer from the gel to a membrane such as nitrocellulose, PVDF, or nylon where they are probed using antibodies specific to the protein. As a result, the analyst is able to examine the amount of protein in a given sample and compare levels between distinct groups of proteins. In the practice of this technique, a sheet of gel is retained between two plates and usually is mounted vertically with the upper edge of the gel sheet accessible to the sample to be assayed. The sample is applied in wells created along the upper edge of the gel and an electrophoretic potential is applied between the upper and lower edges of the sheet of gel. The electrophoretic potential is applied by a DC power supply and may be in the range of 50 to more than 1000 volts. The electrophoretic potential is applied for a period of time that allows the proteins in the sample to distribute themselves (i.e., separate) vertically through the sheet of gel, typically for 1-4 hours, but in some cases considerably longer. The proteins in the “lane” under each well are thus separated into distinct bands of different molecular weights. The potential must be removed when the proteins are distributed as desired. In addition to the sample, one lane of the gel is usually reserved for a marker or ladder of a commercially available mixture of proteins of known molecular weights against which the unknown proteins may be compared. The sheet of gel is removed from between its two glass retaining plates and is then placed on a sheet of blotting material such as porous nitrocellulose of length and width dimensions approximately matching those of the sheet of gel, the blotting material having already been soaked in a buffer to hydrate it. Care must be taken at this step to avoid the presence of air bubbles between the gel and the blotting material, which would impede the direct transfer of the distributed proteins from the gel to the blotting material. Two electrode plates are then placed either side of the gel and blotting material, thereby sandwiching the sheets of gel and blotting material between the electrode plates. The electrode plates should preferably apply a uniform electrophoretic field across the thicknesses of the sheets of gel and blotting material. This electrophoretic field, typically 100-500 volts, transfers the proteins from the gel to the blotting material in the same distribution in which they were captured in the gel matrix. This transfer process takes approximately 1-2 hours, but can take as much as overnight for some proteins to be transferred. After the proteins adhere to the blotting material, the blotting material is removed from the sandwich and is washed in a buffer containing one or more blocking agents such as skim milk, bovine serum albumin or tween-20 detergent for 1-4 hours and then is immersed in a solution of protein-specific reporter antibodies. During the immersion the blotting paper is typically agitated by a rocking or circular motion in the plane of the blotting paper. The immersion step typically takes 1-4 hours, but can take overnight or longer for some antibody-protein pairs. Reporter antibody detection can be done with a variety of markers such as optical dyes, radioactive or chromogenic markers, fluorescent dyes or reporter enzymes depending upon the analytical method used. Western blots are described in detail by Towbin H., Staehelin T., and Gordon J., Proc. Nat. Acad. Sci. USA, 76: 4350-4354 (1979), Burnette W. N., Anal. Biochem., 112: 195-203 (1981), and Rybicki & von Wechmar in J. Virol. Methods, vol. 5: 267-278 (1982).

The foregoing describes the use of a Western blot in a mode utilizing detection by a single antibody. More commonly, Western blots are run using both a primary and secondary antibody. In this mode, the primary antibody, usually a mouse or rabbit antibody, binds to the protein of interest on the blotting membrane. The secondary antibody, often a goat antibody, binds to any antibodies produced in the species used to generate the primary antibody. Typically the secondary antibody will be labeled with a detectable marker, such as fluorescent molecules or horseradish peroxidase. Thus, the primary antibody recognizes the target, the secondary antibody recognizes the primary, and the secondary provides a detectable marker. In all other respects this two-antibody approach is similar to the assay described above utilizing only a primary antibody with a detectable marker attached directly to it.

As is apparent from the foregoing description, the Western blot takes a substantial amount of time to complete and involves a great deal of handling and transfer of materials. This enables variations to creep into the process and its results; the technique is thus dependent to a certain degree upon the skill of the technicians involved. Furthermore, contrary to theoretical prediction, an excessive number of bands may manifest themselves in the result. This can be due to antibodies which are not entirely specific to the protein or proteins of interest, but may also result from other factors. Proteolytic breakdown of the antigen may occur as a result of prolonged storage after homogenization of the starting tissue, resulting in additional bands of lower apparent molecular mass than the full-length proteins. Excessive overloading of protein in a lane may result in “ghost bands” appearing in the blot. High detection sensitivity can give rise to artifacts from nonspecific binding. Inefficient blocking can allow extra bands to develop. A low antigen concentration in the sample can result in poor signal detection requiring signal enhancement, which can introduce its own artifacts. In a reference lab environment, where speed and accuracy are of paramount concern, it is apparent that a more rapid, less technique-dependent definitive test for proteins is desirable.

In accordance with the principles of the present invention a protein assay suitable for use in a reference lab is provided. In an example described below one or more analytes comprising known proteins are resolved in a fluid path such as that of a capillary and the analytes are immobilized in the fluid path. A typical analyte is one or more proteins of a disease condition such as that caused by a virus or by a molecule. A suitable immobilizing technique is photoimmobilization. Patient serum which may contain antibodies to the resolved analytes are then flowed through the fluid path, which will cause binding of patient antibodies, when present, to the known analytes forming, for instance, antibody-protein complexes, which permits detection of the immobilized complexes in the fluid path.

In an example below an automated assay system comprises a processing station and an automated capillary gripper which is operable to load one or more capillaries with one or more reagents or samples and position the loaded capillaries at the processing station. The illustrated automated assay system also includes a detection station and the automated capillary gripper is operable to position the capillaries containing the reagents or samples at a selected location of the detection station.

In the drawings:

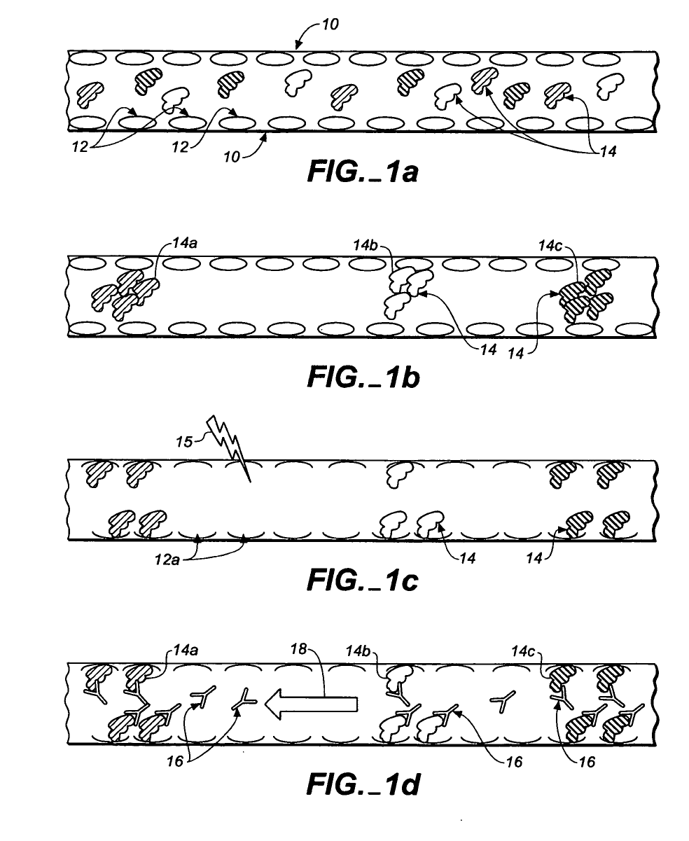

FIGS. 1a-d illustrate an example of resolving, immobilizing and labeling cellular materials in a capillary.

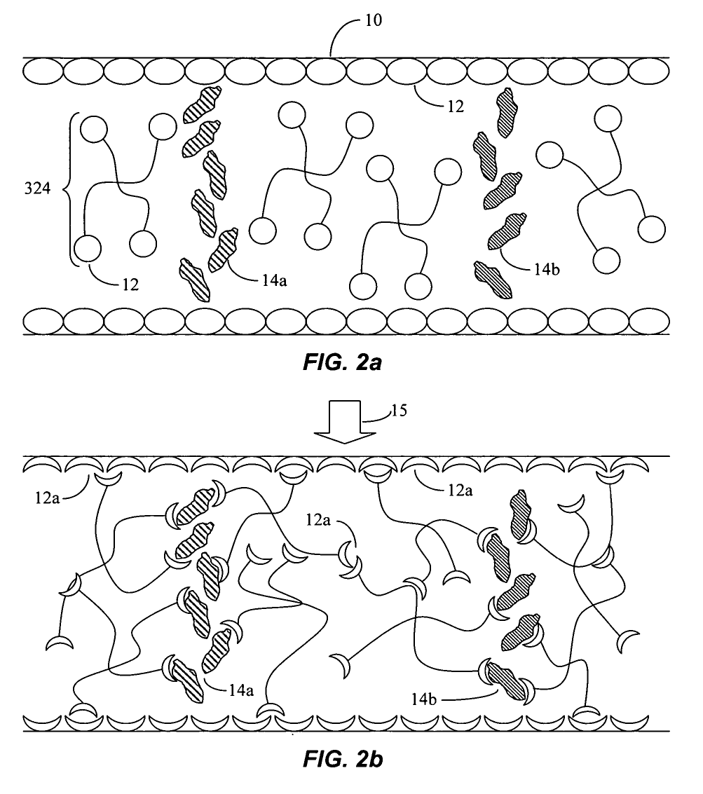

FIGS. 2a-b illustrate an example of immobilizing resolved analytes in a polymeric material in a capillary.

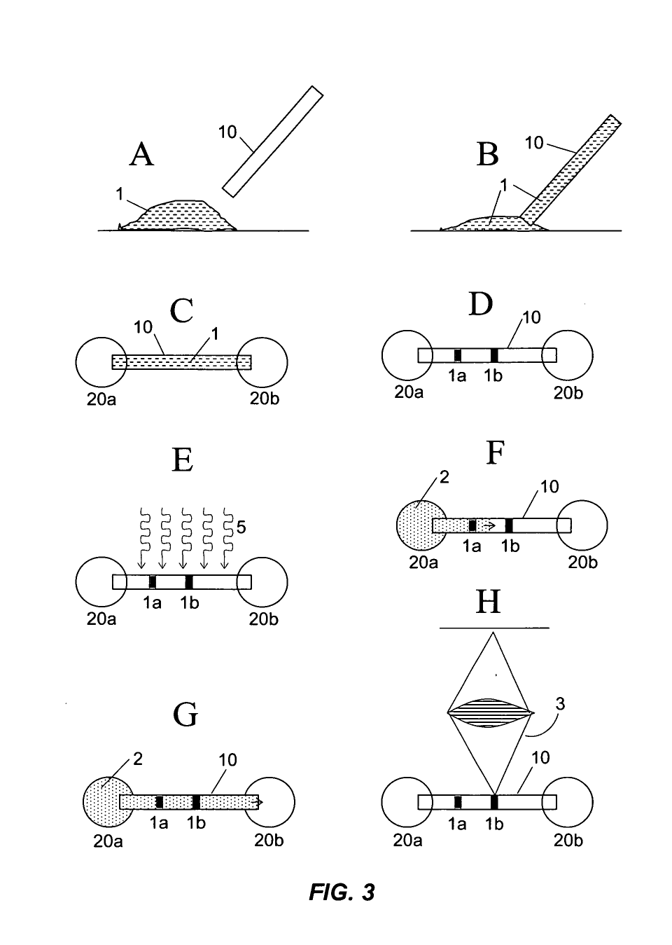

FIGS. 3a-h illustrate an example of detecting one or more analytes.

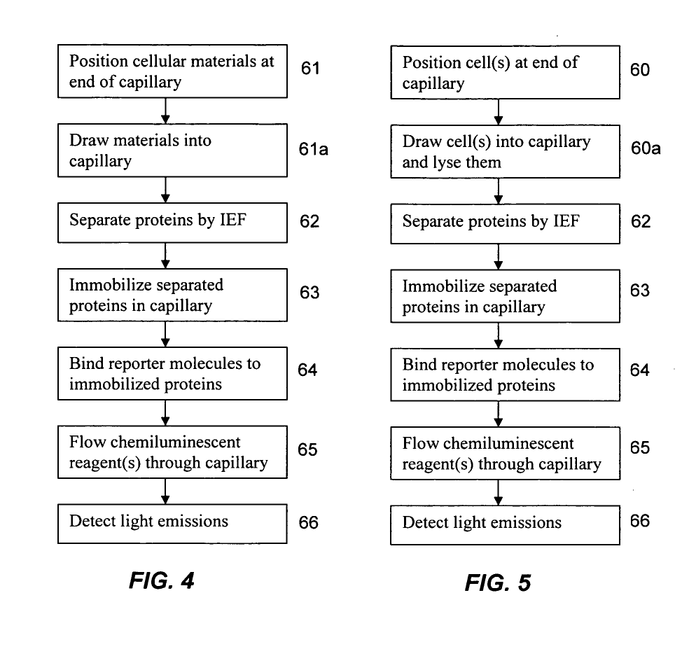

FIG. 4 illustrates an example of detecting cellular materials.

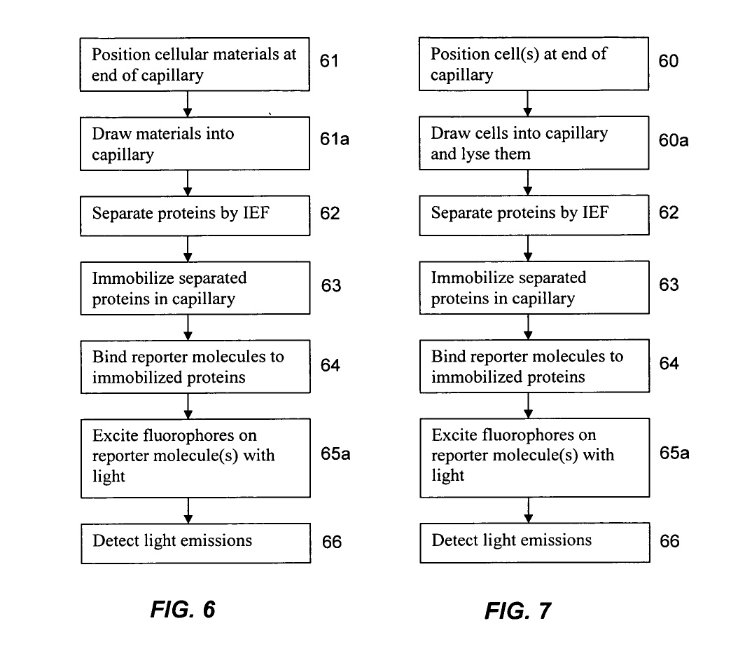

FIG. 5 illustrates an example of analyzing cells.

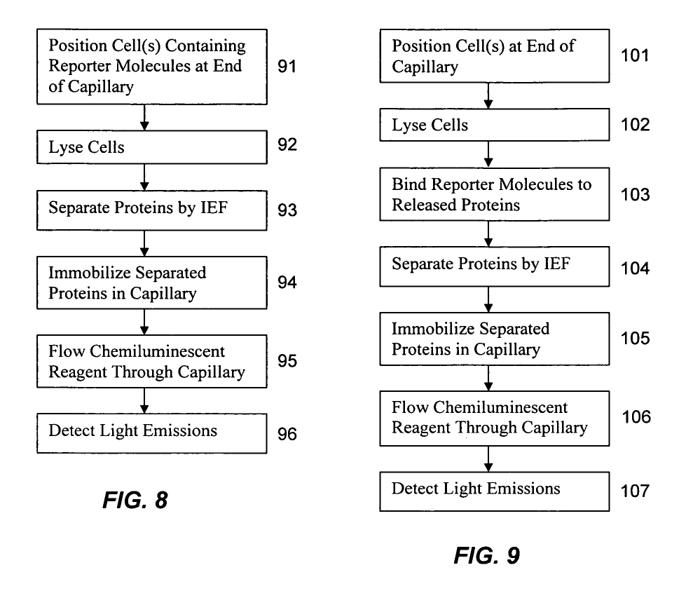

FIG. 6 illustrates another example of detecting cellular materials.

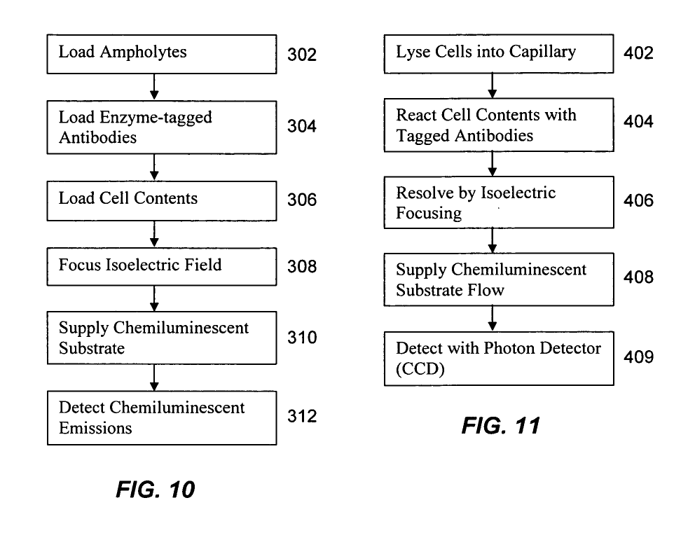

FIG. 7 illustrates another example of analyzing cells.

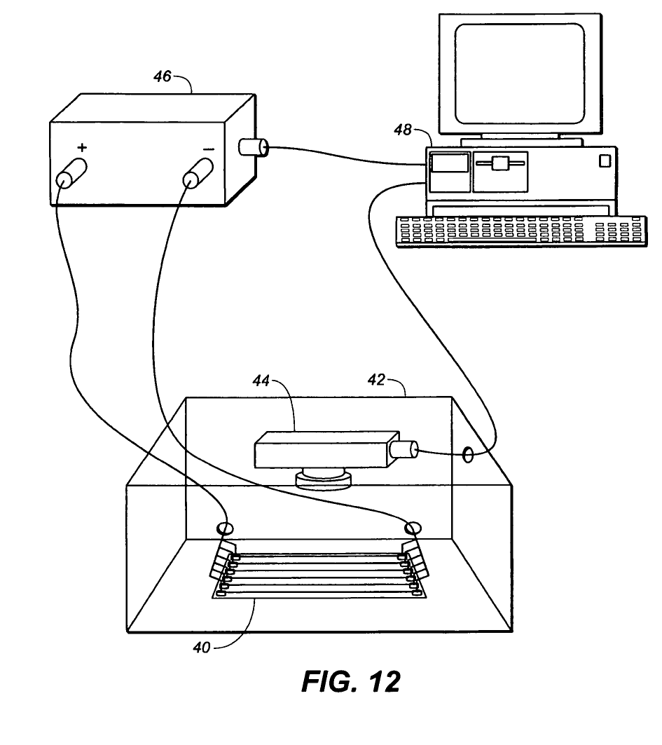

FIG. 8 illustrates another example of analyzing cells.

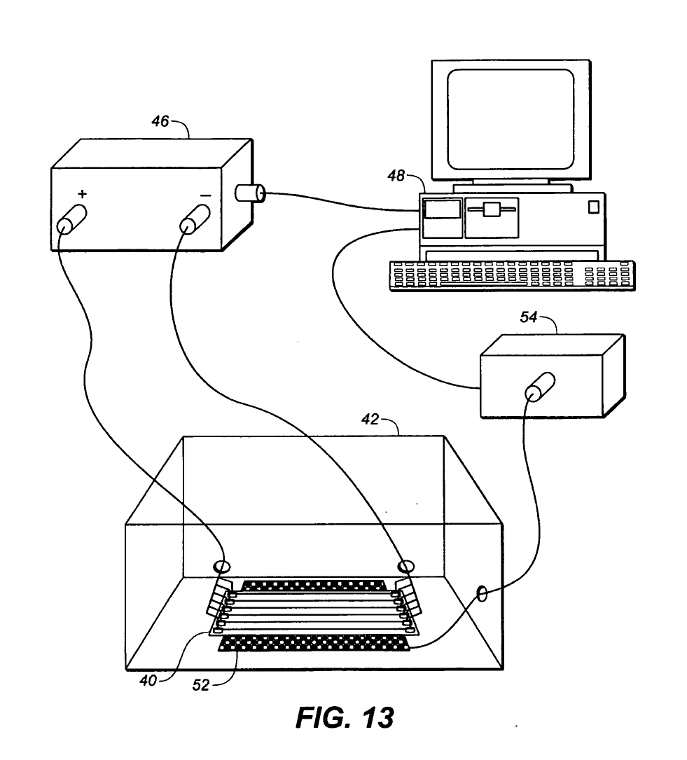

FIG. 9 illustrates another example of analyzing cells.

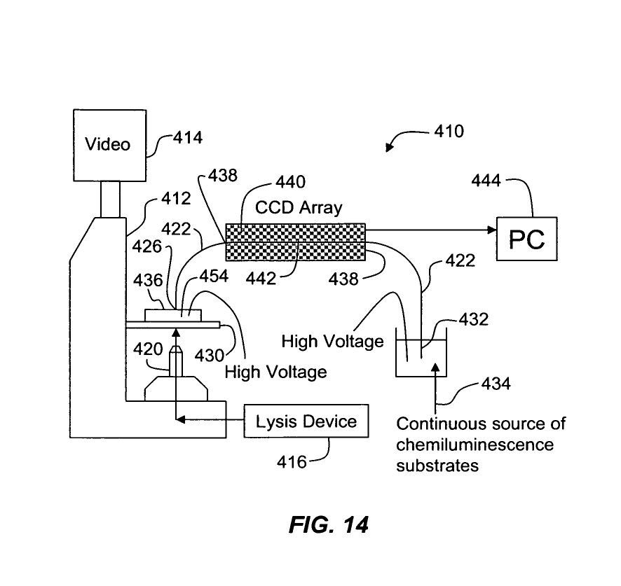

FIG. 10 illustrates an example of analyzing cellular materials.

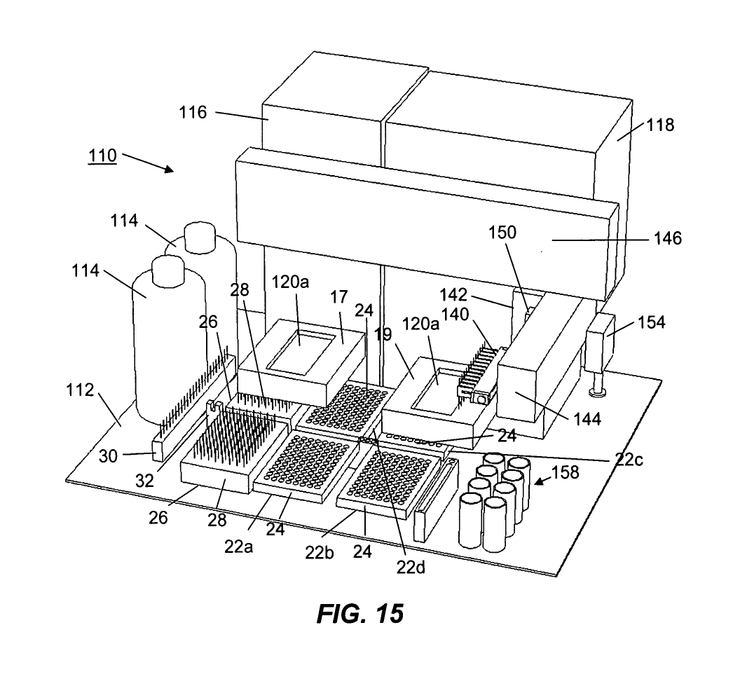

FIG. 11 illustrates an example of a method for analyzing cellular materials.

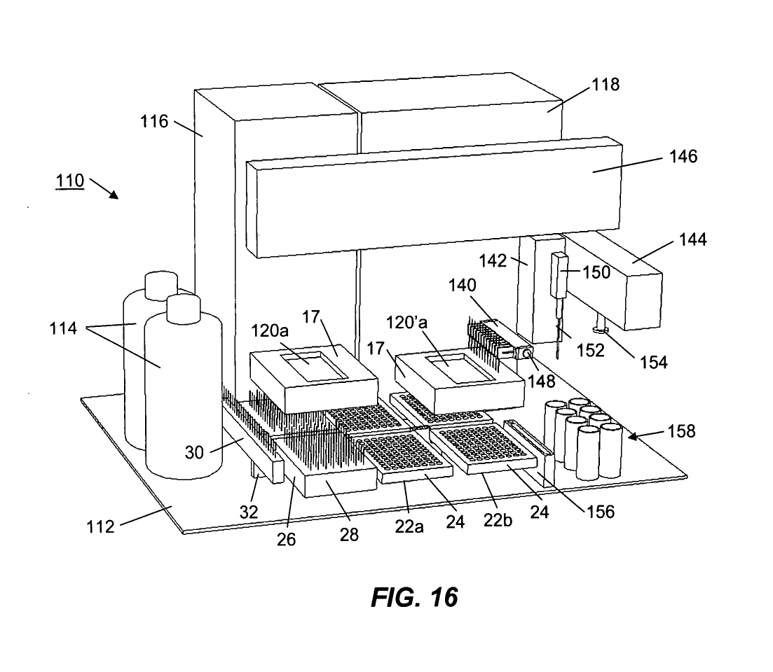

FIG. 12 illustrates an example of an analytical system detection of cellular materials in a capillary by chemiluminescence.

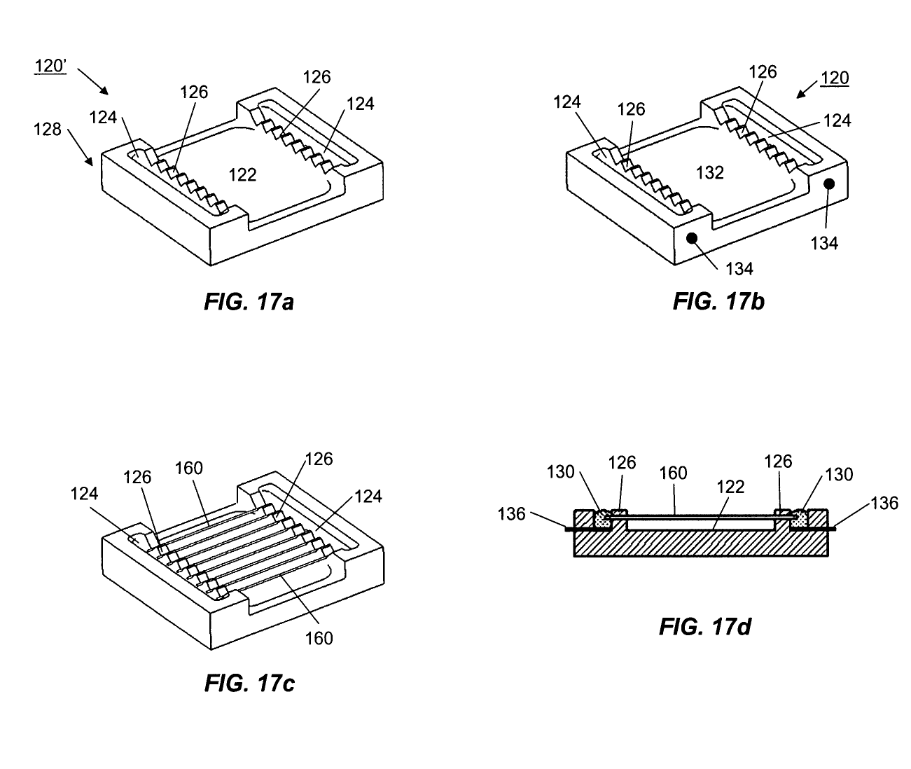

FIG. 13 illustrates another example of an analytical device.

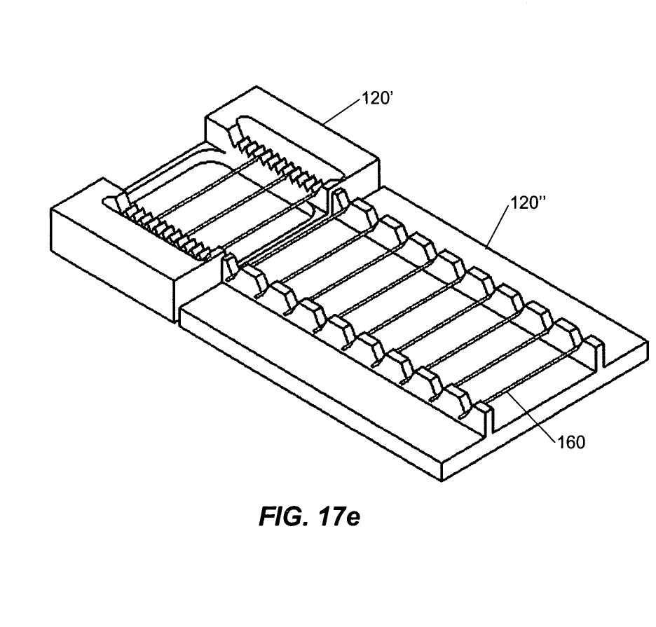

FIG. 14 illustrates another example of an analytical device.

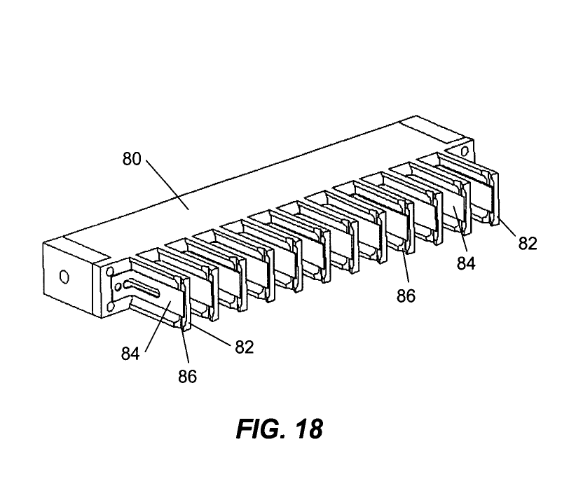

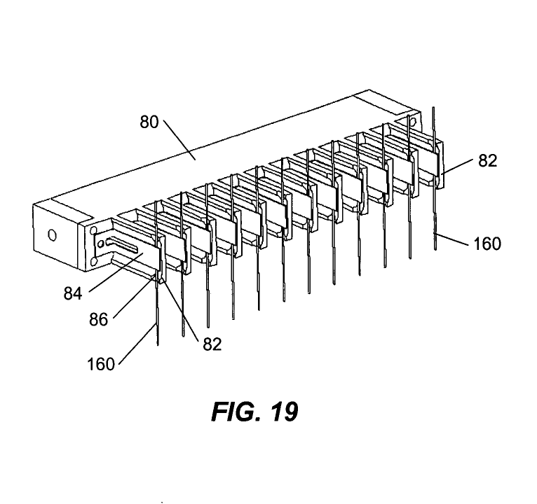

FIGS. 15 and 16 are front perspective views of a micro-volume immunoassay system constructed in accordance with the principles of the present invention.

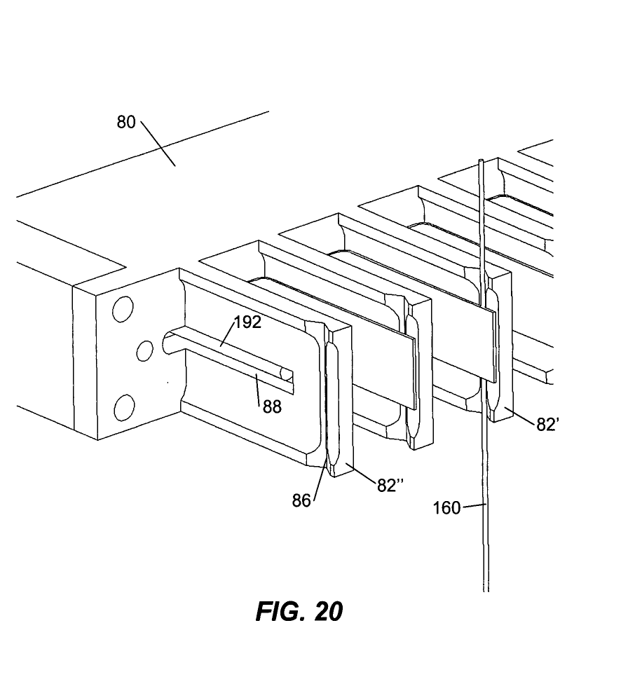

FIGS. 17a, 17b, 17c and 17d illustrate capillary holders constructed in accordance with the principles of the present invention.

FIG. 17e illustrates an intermediate holder for use with a reformatting gripper.

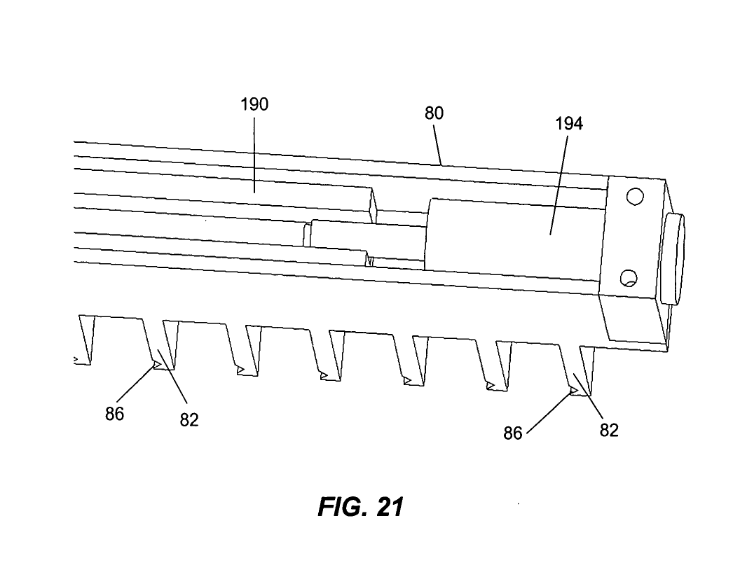

FIG. 18 illustrates a capillary gripper suitable for use in the immunoassay system of FIGS. 15 and 16.

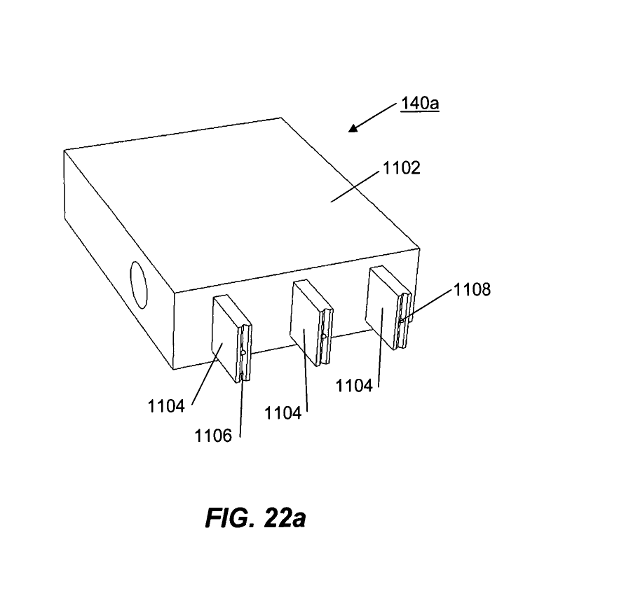

FIG. 19 illustrates the capillary gripper of FIG. 18 when holding twelve capillaries.

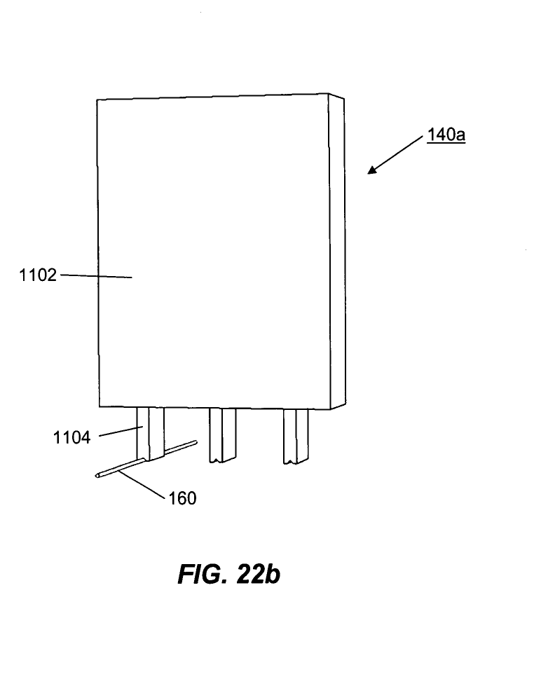

FIG. 20 is an enlarged and cutaway view of the capillary gripper of FIG. 18.

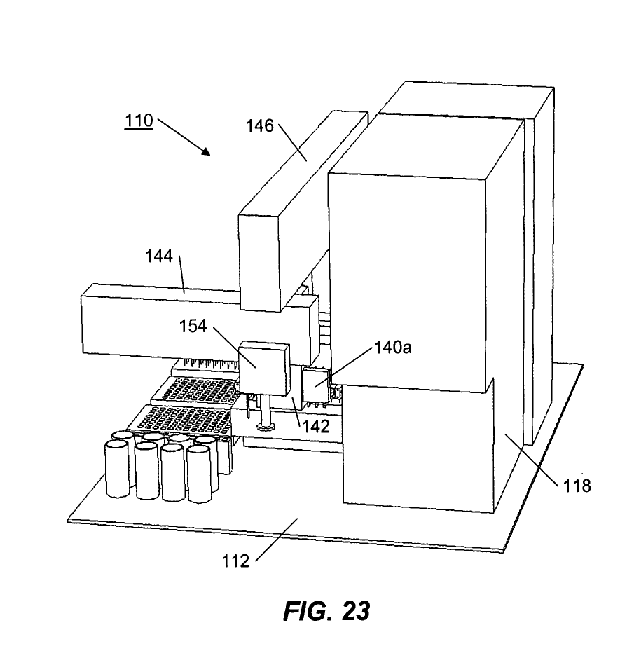

FIG. 21 is a view of the actuator mechanism of the capillary gripper of FIG. 18.

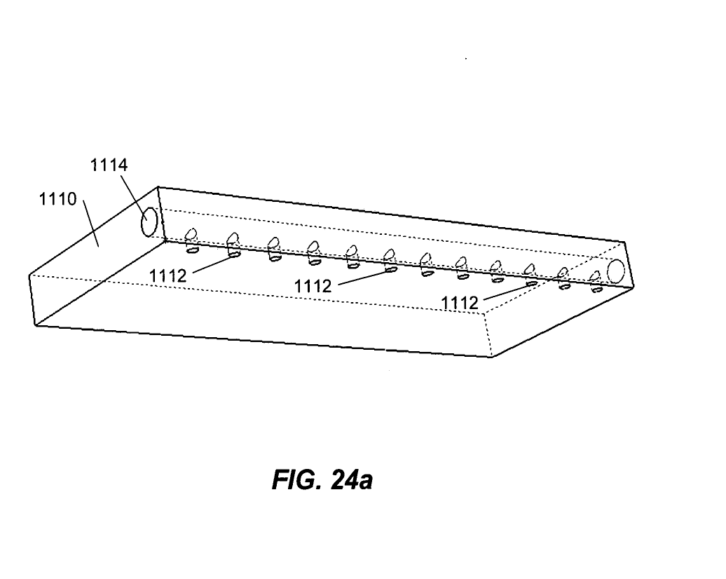

FIG. 22a is a view of a reformatting gripper.

FIG. 22b is a view of the reformatting gripper of FIG. 22a holding a capillary.

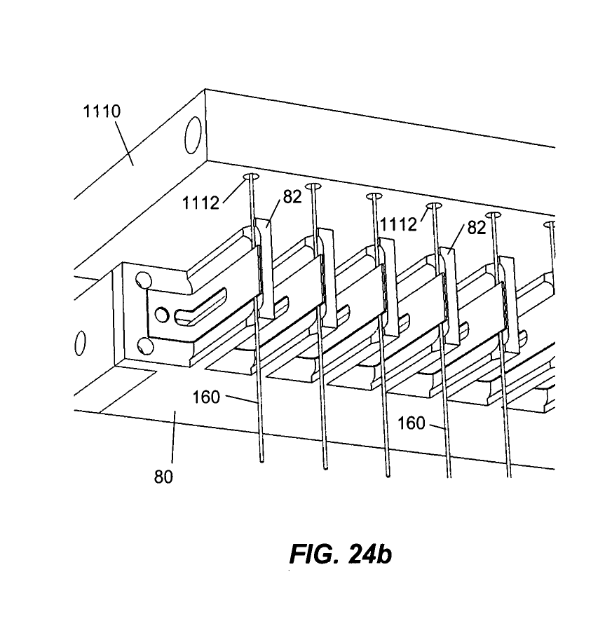

FIG. 23 is a side view of the immunoassay system of FIG. 15 showing the reformatting gripper on a system actuator.

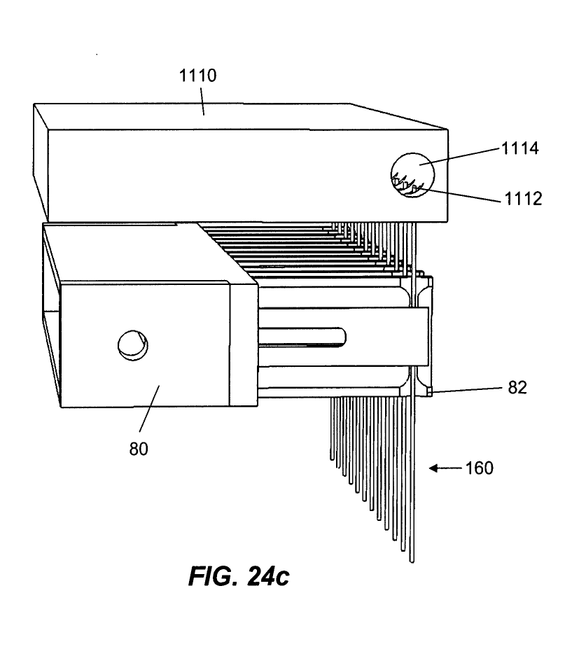

FIG. 24a is a view from below of a vacuum manifold.

FIG. 24b is a view from below of the vacuum manifold of FIG. 24a engaged with capillaries.

FIG. 24c is a side view of the vacuum manifold of FIG. 24a engaged with capillaries.

FIGS. 1a-d illustrate examples of resolving, immobilizing and labeling cellular materials in a capillary in a reference lab assay of the present invention.

FIG. 1a is a longitudinal cross-sectional illustration of a capillary 10 which is lined with a photoreactive group 12. Located within a fluid inside the capillary is a mixture of cellular proteins 14 of differing electrophoretic mobility as indicated by the different shading. In FIG. 1b an electric field has been applied to the fluid to separate the proteins in accordance with their isoelectric points by isoelectrical focusing (IEF) into groups 14a, 14b, and 14c. In FIG. 1c light 15 at the appropriate wavelength is applied to activate the photoreactive group which, when activated as indicated at 12a, binds the proteins 14 at their separated locations within the capillary. Detection antibodies 16 carrying a label are then flowed through the capillary as indicated by arrow 18 in FIG. 1d. The detection antibodies 16 will bind to the proteins 14 they encounter as shown in FIG. 1d. When the detection antibodies contain chemiluminescent label, the bound proteins are then labeled in their bound locations for luminescent detection. In this example, a stream of chemiluminescence reagents can be flowed through the capillary, reacting when encountering the label linked to the proteins. The luminescence from the sites of the proteins is detected by a photon detector and recorded, enabling identification of the proteins by the light emitted from their bound locations. The technique advantageously permits the identification of cellular materials and, in the case where modification of cellular materials (substrates) is being monitored, allows the use of these native substrates without the need to introduce any identification substances prior to the separation of the cellular materials by IEF.

The methods described herein yield results similar to those obtained by a Western blot but in a fraction of the time, significant in the operation of a reference lab. For example, the separation of cellular materials by IEF can take 5 minutes or less, and subsequent immobilization takes 2 minutes or less. This means that the detection molecules can be linked to the separated cellular materials within 10 minutes or less of the commencement of separation, and that the detection molecules can be analyzed within 30 minutes of the separating step. The entire process is faster, simpler, more sensitive, more accurate and more automatable than the Western blot analytical technique. The immobilization step obviates the need to assess the detection molecules (such as enzyme-labeled antibodies) for homogeneity of molecular form prior to use and obviates the need for excessive purification not typical of these types of reagents. Thus, less costly probing antibodies can be used in this technique.

While the separation technique shown in the previous example is isoelectric focusing, free solution electrophoresis, sieving electrophoresis, or micellar electrokinetic chromatography may also be used to resolve the analytes.

FIGS. 2a-b illustrate an example of resolving, immobilizing in a polymeric material, and labeling cellular materials in a capillary in accordance with the present invention. FIG. 2a illustrates a longitudinal cross section of a capillary 10. The upper panel shows capillary 10 walls coated on their interior surface with a photoreactive group 12, represented by closed ovals. A suitable, none limiting example of such material is polyacrylamide containing photoreactive group, such as benzophenone moieties. Also present in the FIG. 2a are polymeric materials 324 in solution, represented by four-armed structures terminated in circles, where the circles represent photoreactive groups 12. A suitable, none limiting example of such material is branched polyethylene glycol bearing photoreactive groups 12 such as benzophenone or ATFB. In addition, two bands of resolved proteins 14a, 14b are shown, represented by the cross-hatched structures. FIG. 2a shows the structure described above after photoactivation 15. Activation of the photoreactive groups is depicted by the concave semicircular structures 12a on both the walls and the polymeric materials filling the capillary. Many of these photoreactive groups 12a are associated with each other, with lengths of polymer, and with the proteins in bands, effectively cross-linking each of these together. Thus, the resolved protein bands are bound in place via a loose network of covalent bonds and polymeric materials. In some implementations, it is desirable that the network form open-pored structures permitting the movement of materials such as detection agents, such as antibodies, through the loose network.

FIGS. 3a-h illustrate exemplary embodiments for detecting one or more analytes in a fluid path. FIG. 3a illustrates a capillary 10 and a sample 1 comprising a mixture of components containing one or more analytes of interest. FIG. 3b illustrates loading the sample 1 into the capillary 10 by capillary action. FIG. 3c illustrates the sample 1 loaded capillary 10, comprising one or more reactive moieties, extending between two fluid-filled wells or troughs 20a, 20b. The components of the sample 1 are separated such that the analyte 1a or analytes 1a and 1b of interests are resolved by one or more electrodes in contact with a solution on one side of the capillary 10 and another one or more electrodes is in contact with a solution on the other side of the capillary 10 as illustrated in FIG. 3d. FIG. 3e illustrates the activation of one or more reactive moieties capable of immobilizing the analytes 1a and 1b of interests in the capillary 10. Detection agents 2 are then flowed through the capillary 10 as indicated by the arrow in FIGS. 3f and 3g. Detection agents 2 are then detected 3, enabling detection of the analyte of interest in their immobilized locations in the capillary by the signal emitted as illustrated in FIG. 3h.

FIG. 4 illustrates an example of a method of the present invention for analyzing cellular materials. In step 61 the cellular materials to be analyzed are located at one end of a capillary. In step 61a the cellular materials are loaded in to the capillary. In step 62 the cellular materials are separated within the capillary, for example by IEF. In step 63 the separated materials are immobilized in the capillary. In step 64 detection agents, for example reporter antibodies, are bound to the immobilized analytes, such as proteins in the capillary. In step 65 a chemiluminescent reagents, or other detection agents, are flowed through the capillary to produce the event to be detected, such as chemiluminescence. The emitted light is then detected in step 66.

FIG. 5 illustrates another example of a method of the present invention for analyzing cells. In step 60 one or more cells to be analyzed are positioned at the end of a capillary. In step 60a one or more cells are drawn into the capillary and are lysed. In step 62 the cellular materials are separated within the capillary, for example by IEF. In step 63 the separated materials are immobilized in the capillary. In step 64 detection agents, for example reporter antibodies, are bound to the immobilized analytes, such as proteins in the capillary. In step 65 a chemiluminescent reagent, or other detection agents, are flowed through the capillary to produce the event to be detected, such as chemiluminescence. The emitted light is then detected in step 66.

FIG. 6 illustrates another example of a method of the present invention for analyzing cellular materials. In step 61 the cellular materials to be analyzed are located at one end of a capillary. In step 61a the cellular materials are loaded into the capillary. In step 62 the cellular materials are separated within the capillary, for example by IEF. In step 63 the separated materials are immobilized in the capillary. In step 64 detection agents, for example reporter antibodies, are bound to the immobilized analytes, such as proteins in the capillary. In step 65a fluorophores on the detection agents, for example fluorescent labeled antibodies, are excited with light. The emitted light is then detected in step 66.

FIG. 7 illustrates another example of a method of the present invention for analyzing cells. In step 60 one or more cells to be analyzed are positioned at the end of a capillary. In step 60a one or more cells are drawn into the capillary and are lysed. In step 62 the cellular materials are separated within the capillary, for example proteins are resolved by IEF. In step 63 the separated materials are immobilized in the capillary. In step 64 detection agents, for example reporter antibodies, are bound to the immobilized analytes, such as proteins in the capillary. In step 65a fluorophores on the detection agents, for example fluorescent labeled antibodies, are exited with light. The emitted light is then detected in step 66.

FIG. 8 illustrates an example of the present invention in which labeled cellular materials are released from the cell at the moment of their introduction into the capillary. The cellular materials are then separated and immobilized. In step 91 one or more cells containing detection agents are located at one end of a capillary. The cell or cells are then lysed in step 92 to release their labeled proteins and transported in the capillary. In step 93 the cellular materials are separated, for example within the capillary by EF. In step 94 the separated materials are immobilized in the capillary. In step 95 a chemiluminescent reagent is then flowed through the capillary to produce photons by chemiluminescence. The emitted photons are then detected in step 96.

FIG. 9 illustrates an example of the present invention in which analytes are labeled prior to separation. In step 101 one or more cells are located at one end of a capillary. The cell or cells are then lysed in step 102 to release their contents. In step 103 detection agents are bound to the released cellular contents, for example proteins. In step 104 the cellular materials are separated within the capillary by IEF. In step 105 the separated labeled materials are immobilized in place in the capillary. In step 106 a chemiluminescence substrate is then flowed through the capillary to produce photons by chemiluminescence. The emitted photons are then detected in step 107

FIG. 10 illustrates an example of the present invention for chemiluminescent detection of analytes. In step 302 an ampholyte reagent for the pH gradient is loaded into the fluid path. In step 304 enzyme-labeled antibodies able to catalyze chemiluminescence and able to bind the analyte of interest are loaded into the fluid path. The cell contents are loaded into the fluid path in step 306, whereupon the enzyme-tagged antibodies will bind with the analyte or analytes of interest. A focusing isoelectric field is applied in step 308 to resolve and then immobilize the enzyme-tagged antibodies and analytes in a pH gradient. A chemiluminescent substrate compatible with the enzyme-labeled antibodies is supplied in step 310 and chemiluminescent emissions are then detected from the interaction of the chemiluminescent substrate with the enzyme-labeled antibodies and bound to analyte in step 312. In some implementations, the analyte is immobilized by IEF and the chemiluminescent reagent is flowed through the fluid path by carryings it's own charge at all pH's of the gradient.

FIG. 11 illustrates an example of the present invention for chemiluminescent detection of analytes. In this example, a cell is lysed into or at the inlet of a capillary in step 402. The lysis releases cellular contents which react with detection agents, for example chemiluminescent labeled antibodies, in step 404. The labeled and bound cellular contents are resolved in the capillary by isoelectric focusing in step 406. In step 408 a chemiluminescent reagent is supplied which will react with the enzyme of the antibodies bound to the cellular contents. In this example, the analyte is immobilized by IEF and the chemiluminescent reagent is flown through the fluid path by carryings it's own charge at all pH's of the gradient. In step 409 chemiluminescence is detected with a photon detector such as a photocell or CCD array detector.

Variations of the steps of the methods described herein will readily occur to those skilled in the art. For example, the sample can be separated and then the analytes immobilized at their resolved locations in the fluid path, prior to contacting the analytes with the detection agents. In some implementations, detection agents are contacted with the analytes to form a complex and then the complex is resolved in the fluid path. In some implementations, the detection agents is preloaded into the sample thereafter loaded into the system. As another example, the resolving step, such as isoelectric focusing can be applied after the chemiluminescent reagents are supplied.

FIG. 12 illustrates an exemplary analytical device constructed in accordance with the principles of the present invention. An array of capillaries 40 which are loaded with the necessary one or more reactive moieties to immobilize the analytes, buffer, and sample to be analyzed is located in a light-tight box 42. A controllable power supply 46 is coupled to the electrodes on either end of the capillaries to apply the voltages needed to separate the sample and to flow the detection agents and/or chemiluminescent reagents through the capillaries. A voltage is applied to flow the sample into the capillary and to separate the sample, for example by isoelectric focusing. Alternatively, the sample may be loaded into the capillary by hydrodynamic flow and thereafter separated, for example by isoelectric focusing. An energy source (not shown) capable of activating the reactive moieties is provided. For example, a light source such as an ultraviolet lamp provides illumination inside the box to immobilize the individual components of the sample in their separated locations. In some implementations, the system comprises a light source for induction of fluorescent label. One or more detection agents, such as those described herein, are introduced into the wells at one end of the capillaries and flowed through the capillaries, binding to the analytes. In some implementations, detection reagent is introduced into the wells and flowed through the capillaries. Detection agents may be introduced from separate smaller wells if desired. Additional smaller wells can be used to conserve detection agents. Viewing the capillaries within the box 42 to receive the photons emitted from the immobilized analytes and detection molecules is a CCD camera 44. The system is controlled by a computer 48 which switches the power supply 46 and the light, controls the application of detection molecules and reagents, and records and analyzes the photon signals received by the CCD camera 44. Similarly, a light source for induction of fluorescence of molecular standards run in the separation may allow detection with the same CCD camera used to detect chemiluminescence-produced light. Internal standards serve to calibrate the separation with respect to isoelectric point, or for an alternative separation mode, molecular weight. Internal standards for IEF are well know in the art, for example see, Shimura, K., Kamiya, K., Matsumoto, H., and K. Kasai 10 (2002) Fluorescence-Labeled Peptide pI Markers for Capillary Isoelectric Focusing, Analytical Chemistry v74 at 1046-1053, and U.S. Pat. No. 5,866,683. Standards to be detected by fluorescence could be illuminated either before or after chemiluminescence, but generally not at the same time as chemiluminescence.

In some implementations, the analyte and standards are detected by fluorescence. The analyte and standards can each be labeled with fluorescent dyes that are each detectable at discrete emission wavelengths, such that the analyte and standards are independently detectable.

FIG. 13 is an exemplary system in which the photons emitted from the detection molecules are received by a CCD array located beneath the capillary array 40. The CCD array 52 is monitored by a CCD controller 54 which provides amplified received signals to the computer 48.

FIG. 14 illustrates an exemplary system of an analytical device for capillary detection of cellular material by chemiluminescence. The system 410 comprises a microscope 412 having a video ocular readout 414 such as a CCD camera displayed on a CRT screen and/or recorded by a videotape recorder or digital recorder (not shown). In some implementations, the system allows digital storage of the images and pattern processing in a computer system for automated cell processing and analysis. The CCD video camera system 414 is capable of recording a real time bright field image of a target cell. The device may optionally comprise a cell lysis device 416, such as a laser, sonic generator, electronic pulse generator, or electrodes positioned adjacent to target cell(s) on a cover slip 436. In the illustrated example, after cell lysis, cell contents are loaded into the end of the capillary by hydrodynamic flow or electrophoresis. In some implementations, this end of the capillary has already been loaded with a short (few mm or less) slug of labeled antibodies at the time of cell lysis. Thereafter, following a hybridization period, if necessary, separation for example by isoelectric focusing is initiated.

A fused silica capillary 422 is positioned with a micromanipulator (not shown) so that the inlet 426 of capillary 422 is located above the cover slip 436 or slide or microwell plate performed by loading the cell with detection agents prior to lysis or hybridization may be performed in the buffer solution subsequent to lysis. In the latter event, a high concentration of detection agents surrounds or is located adjacent to the cell. One method for achieving the desired high concentration of cell contents in contact with a high concentration of detection agents is to draw the cell contents by hydrodynamic or electrophoretic means into a short length of the capillary adjacent to the capillary end. In this mode this short region of the capillary may be pre-loaded with detection agents from another source such as a tube or well (not shown), or may be drawn into the capillary end along with the cell contents. The distal end 432 (or proximal end 426) of capillary 422 is disposed in a solution 434 of chemiluminescent substrates. In some implementations, resolving and immobilizing the analyte or analytes of interest can occur prior to adding the detection agents. The detection agents are then flowed though the fluid path after the separating the sample and immobilization.

Ampholyte reagent 442 is electrically coupled to a high voltage potential which, when applied to the capillary solution, causes the development of a pH gradient within the capillary 422 by ampholyte migration. A high voltage power supply, such as model CZE 1000R manufactured by Spellman of Plainview, N.Y., which is capable of providing a 20,000 volt potential can be used to maintain the pH gradient in column or capillary 422.

Fused silica capillary 422 may typically exhibit a 100 micron inner diameter and 360 micron outer diameter. The lumen walls can be coated with a neutral coating such as that manufactured by Supelco of Phoenix, Ariz. The coating is used to minimize the electroosmotic flow and thus shorten the migration times for the antibody-target complexes. The total length of the capillary in this example can be as long as 90 to 100 cm, but preferably is considerably shorter, in the range of 10 to 30 cm, or 3 to 6 cm. The cell chamber 436 serves as an inlet reservoir for targeted cell molecules and optionally the ampholyte reagent and chemiluminescent reagent(s) and can be held at ground potential relative to the high voltage potential at the other end of the capillary. In some implementations either end of the capillary may be at high voltage potential and the other ground, or either end may be positive and the other negative. The outlet reservoir 434 may be held at 15 to 20 kV relative to ground at the proximal end of the capillary, for example. The actual potential used is generally chosen by the desired voltage drop per cm of capillary. Distal outlet 432 of capillary 422 is placed about 5 centimeters below inlet 426 in the case of hydrodynamic loading. For the case of electrophoretic loading, which may be equally or more effective, no particular elevation of the distal end of the capillary is required. Inlet 426 of capillary 422 is used as a micropipette for introducing the cellular contents into capillary 422 after cell lysis. Alternatively, the cell may be drawn intact into the capillary and then lysed in the capillary.

After removing 5 mm of polyimide coating from capillary 422 above inlet 426, inlet 426 is mounted perpendicular to cover slip 436 by a micromanipulator (not shown). The micromanipulator enables precise positioning of the capillary lumen with respect to the target cell to be loaded or lysed and loaded into capillary 422.

Capillary 422 includes an optical observation window 138 through which chemiluminescent or fluorescence events are observed and detected by a CCD array 440 or similar detector. An extended observation window 438 is desirable as it enables the parallel detection of a greater number of events than can be observed through the limited length of a shorter window. Generally the length of the observation window will be chosen in consideration of the length of the CCD array 440 being used. If a non-clear coated capillary is used the polyimide coating of capillary 422 is removed over at least the length of the capillary which opposes the CCD array 440. The observation window 438 is maintained in a fixed position in relation to the CCD array 440 either by mechanical or adhesive means. Preferably the observation window and CCD array are enclosed in darkness so that the only light detected by the CCD array is that emitted by the chemiluminescent or fluorescent events within the capillary. The signals from the detected chemiluminescent or fluorescent events are coupled to a personal computer 444 where they are recorded. In some embodiments the event data may be recorded along with the position in the CCD array at which the event occurred. The data is plotted and total signal corresponding to each focused band calculated using Origin software available from Microcal of North Hampton, Mass., DAX software available from Van Mierlo, Inc. of Eindhoven, The Netherlands, LabVIEW software available from National Instruments Corp., Austin, Tex., or similar data analysis packages. Data may be presented as a histogram, electropherogram, or other graphical representation, or as a spreadsheet or other numerical format.

In some implementations, a cell or cells which have not been preloaded with detection agents, the inlet 426 of the capillary 422 is positioned directly above the target cell or cells to be lysed. The cell or cells can be in contact with a high concentration of detection agents, or preferably, a high concentration of detection agents has already been loaded into the capillary end at the time of cell lysis. The lysis device 416 is aimed to create a lysing shock wave or other cell lysing disruption adjacent to the cell or cells. When the lysing pulse is applied the cellular contents are released and the force of the lysing event may aid in propelling the cellular contents into the lumen of the capillary by hydrodynamic flow, electrophoresis, or electroendosmotic flow. Hybridization of the analytes of interest and the detection agents takes place rapidly, either outside the capillary prior to loading of the cell contents, or inside the capillary. The degree of hybridization will be linearly related to the concentrations of the detection agent and the sample. For example, tight-binding (high binding avidity) antibodies provide molecules which will retain their linking characteristics during capillary transport and isoelectric focusing. Examples of such antibodies are those typically used in ELISA assays. Preferably the hybridization is done under non-denaturing conditions. By causing the antibodies and their analytes to be in a natural state, recognition between the antibodies and their target complexes and the chemiluminescent reporters is enhanced. The isoelectric focusing field is applied, causing the antibody-target complexes to migrate to pH points of the pH gradient in the capillary at which their net charge is neutral. The complexes will become stationary in the capillary at pH points where the charge of their molecular components (e.g., phospho, carboxyl, amino, and other charged functional groups) nets out to zero. If forces from flow or diffusion should cause the complexes to drift away from their respective isoelectric points, the gradient field will migrate them back into their charge-neutral positions. The antibody-target complexes are thus resolved along the observation window 138 by capillary isoelectric focusing. In some embodiments, resolving and immobilizing the analyte or analytes of interest can occur prior to adding the detection agents. The detection agents are then flowed though the fluid path after the separating the sample and immobilization.

The electrophoretic potential is then used to cause the chemiluminescent substrate solution 434 to flow through the capillary. This may be initiated at the same time as the electric field which is first applied to establish the pH gradient, or after the gradient has already been established and the antibody-target complexes have been focused. The substrate or substrate(s) are chosen such that they exhibit(s) a net charge at all pH conditions encountered within the capillary so that the substrates do not resolve within the capillary but continue to flow in a continuous stream. As the substrates encounter antibody-target complexes along the capillary they are cleaved by the reporter enzyme of the antibody of the complexes, causing release of photons. Thus, as the stream of chemiluminescent substrate continuously flows through the capillary, the resolved antibody-target complexes will continue to emit photons. Alternatively, an excitation source may be used allowing fluorescence detection. In embodiments where chemiluminescence is used, emission is continuous for as long as the flow of chemiluminescent substrates is promoted, and the noise associated with stray excitation light in fluorescence-based systems is avoided.

The photon emission events are detected by the adjacent CCD array 440 and the detected events accumulated by the computer. Detection and accumulation can be continued for a selected period of time, enabling long detection periods to be used for sensitive detection of very small amounts of targeted cellular molecules. When only a single labeled antibody is used, the number of events accumulated will be a measure of the amount of analytes in the cell or cells used to prepare the lysate. To measure the amounts of different cell proteins or molecules, different antibodies which create different antibody-target complexes at different isoelectric points can be employed. By recording the number of photon events and the locations along the CCD array at which the events were detected (corresponding to the focused bands or isoelectric points along the gradient field of the capillary) the photon events emanating from the differently labeled analytes can be segmented. For increased throughput, multiple parallel capillaries or channels can be run past one or more CCD arrays incorporated into a single instrument. In another implementations, multiple antibodies labeled with different fluorescent dyes having spectrally resolvable signals can be used to enable multiplexed analysis of different proteins in a single capillary.

Fluorescent standards can be read separately if desired, using the same detector before or after the chemiluminescence signals have been collected, by exposing the fluors to excitation light. For an all-fluorescence system, the analyte and standards can be discerned by using differentially excitable and detectable dyes.

While the CCD array is preferred for its ability to detect in parallel the photon events occurring along the array, it is understood that more restricted detection techniques may be acceptable in a given embodiment. For instance, a single photon sensor may be swept or moved along the observation window 438 to detect the chemiluminescent or fluorescent photon events. This approach, however, may miss an event at one point of the capillary when the sensor is aimed or located at a different point of the capillary. Furthermore by the use of a single sensor designed for commonly available fixed window location capillary electrophoresis instruments, resolution can be deteriorated by laminar flow within the capillary, and chemiluminescent or fluorescent sensitivity would be reduced due to the limited time that a photon source is in the observation window.

Further details of the methods and apparatus of the invention described above can be found in U.S. patent application Ser. No. 11/185,247, filed Jul. 19, 2005, the contents of which are hereby incorporated by reference.

An automated protein assay system suitable for use in a reference lab is shown in FIGS. 15-24 below. Referring first to FIGS. 15 and 16 a micro-volume immunoassay system 110 constructed in accordance with the principles of the present invention is shown in a perspective view. The system 110 is mounted on a baseplate 112 with a surface suitable for wipe down if biohazardous materials are processed though the system (e.g., stainless steel or plastic). Shown on the baseplate 112 are a pair of bottles 114 which contain bulk fluids. Clean wash fluid is pumped for use from one bottle while all system waste fluids are pumped into the other bottle. Toward the back of the baseplate 112 is a detection module 116. The detection module 116 houses a movable tray 17 with a space 120a for a capillary holder 120′ (shown in FIG. 17a) and an intermediate holder 120″ (shown in FIG. 17e). The movable tray 17 is automated to slide into and out from the detection module in the manner of a disk tray of a computer optical disk drive to transport the capillary holders into and out of the module 116. The detection module houses an imaging optical detector for detecting light emitted from within the capillaries. In this example the optical detector is a cooled charge-coupled device (CCD) array detector. Light from the capillaries is imaged onto the CCD by a lens assembly. The detection module is light-tight when the tray 17 is retracted to the interior of the module, enabling the CCD array detector to detect light emitted from a capillary by chemiluminescence or fluorescence. To excite fluorescence an array of light emitting diodes inside the detection module is arranged to uniformly illuminate the capillaries. Alternatively, a laser or other light source could be used for excitation. Wavelength-selective filters are used to prevent excitation light, emitted from the light emitting diodes, from interfering with detection of the fluorescent emissions. For chemiluminescent detection a material such as luminol is flowed through the capillaries by hydrodynamic flow as described below and the emitted photons are detected by the CCD array detector. In an alternative implementation luminol may be electrically pumped through the capillaries by application of voltage across the capillaries with hardware similar to that described in the section below for isoelectric focusing. During detection the capillary is held in a capillary holder 120′ on the tray 17 which is retracted into the detection module and moves out again after photodetection is completed. The detection process may take from seconds to hours depending on the level of sensitivity desired. The capillary holder is described in greater detail in FIGS. 17a-17e. The capillary holder may hold a plurality of capillaries so that the CCD array will detect photoemissions from a plurality of capillaries at the same time. In an alternate implementation a scanning fluorescence detector may be used. In that implementation excitation light focused by a lens irradiates fluorescent molecules within each capillary. This same (or another) lens collects the resulting fluorescent emission for detection by a photo sensitive device such as a photo-multiplier tube. This focused excitation/collection can be scanned along the length of each capillary individually or in groups. The excitation light is a coherent source such as a laser or an incoherent source such as an arc lamp or light emitting diode array.

Adjacent to the detection module 116 is a separation and immobilization module 118. This module contains a movable tray 19 with a space 120′a for a capillary holder 120′ and intermediate holder 120″ and the electronics for conducting electrophoresis and isoelectric focusing of substances in the capillaries when the capillaries are located in a capillary holder 120′. As described below, the capillary holder 120′ has two integrated electrodes that are electrically connected to respective fluid reservoirs on opposite sides of the capillary holder. The ends of the capillaries (see FIG. 17c) in the capillary holder are in fluidic contact with the fluids in these two reservoirs. Thus, when the separation and immobilization module 118 applies a voltage across the electrodes, this voltage is applied across the fluid paths within the capillaries in the capillary holder 120′. The voltage applied to the capillaries is regulated by a computer controlled power supply located inside or coupled to the module 118. This voltage causes the biological molecules to separate by isoelectric focusing. In an alternate implementation, the molecules may be separated by size or other techniques. Following separation of the biological molecules in the fluid paths of the capillaries in accordance with their ionic charge by isoelectric focusing, the separated molecules are immobilized in their focused positions in the capillaries. In the illustrated example this is accomplished by irradiating the capillaries with ultraviolet light from a UV light source inside the separation and immobilization module to bind the separated material to either a material within the lumen or to the walls of the capillaries by photoactivated chemistry.

Typically 1-1000 mW/cm2 for 1-200 s UV light is used. The isoelectrically focused materials are thereafter detected in the detection module 116. It may be desirable in some situations to retain the capillary holder 120′ in a level position to minimize hydrodynamic flow through the capillaries during focusing and immobilization. Alternatively, increasing fluid viscosity will reduce fluid flow. Yet another approach is to incorporate a small connecting fluid path (e.g., a channel) between the two reservoirs of the capillary holder. This path would typically be about 1 mm across, so that the fluid heights between the two reservoirs will quickly equalize.

Located on the baseplate 112 in this example are a number of microwell plate stations 22a-22d. In the illustrations of FIGS. 15 and 16 there is a microwell plate 24 located in each of the stations 22a-22d. Preferably the microwell plate(s) containing samples are chilled while in these stations. This may be accomplished preferably by thermoelectric cooling or by other means such as refrigeration, recirculating cold fluid or an ice bath coupled to the sample plates. The stations have guides or recesses which precisely define the locations of standard microwell plates when located in the stations. Each microwell plate station in this example is marked on the baseplate 112 by a distinguishing color or graphic which is visible through a translucent microwell plate, enabling each station to be distinctively identified as discussed below. A standard microwell plate may contain 96 microwells on a 9 mm center-to-center spacing or 384 microwells on a 4.5 mm center-to-center spacing. Plates with other spacings and numbers of wells may be used. This enables a robotic, computer-controlled capillary manipulator as described herein to be able to access a preselected well in a microwell plate 24 since the plates and each of their wells are in specific, predefined positions on stations 22a-22d. Computer control enables the specification of the microwell plate to be chosen from several predetermined standards to which the capillary manipulator is programmed. Also located on the baseplate 112 in this example are a pair of bulk capillary rack stations 26. As in the case of the microwell plate stations, the capillary rack stations locate standardized capillary racks 28 in predetermined locations so that capillaries in the racks 28 can be automatically accessed by a robotic computer-controlled capillary manipulator to pick up capillaries from the racks 28. A capillary rack 28 may contain 96 capillaries on a 9 mm center-to-center spacing or 384 capillaries on a 4.5 mm center-to-center spacing. If the capillaries utilize an internal wall coating for immobilization, they may be supplied precoated in the racks.

Capillaries are preferably made from a transparent low fluorescence material such as glass that is also rigid and straight. Various inside diameters (typically 10 μm to 1 mm) and lengths (typically 30 mm to 100 mm) are commonly used. In one example, a capillary is 40 mm in length with an internal diameter of 100 μm, giving the capillary a volume of 314 microliters. Various cross sectional shapes, both inside and outside, are also possible. One could also use different materials such as plastic.

In an alternative implementation a microfabricated device may be used in place of individual capillaries or a combination thereof. These microfabricated devices are fabricated with internal capillary channels whose dimensions would be similar to those described previously for capillaries. A microfabricated device can be fabricated from various materials such as silicon, glass or plastic and may contain integrated electrodes, electronics and valves. They may be disposable or re-usable devices. Microfabricated devices can contain from one to hundreds of channels that can be controllable individually or in parallel or some combination thereof. A typical microfabricated device contains wells for adding samples or other reagents. External electrodes may also be inserted into these wells. As with capillaries, the cross section of a capillary channel is not constrained to any particular shape.

In the illustrated example capillaries are removed from a storage rack 28 of, for example, ninety-six capillaries, by a robotic, computer-controlled capillary manipulator and placed prior to use into a capillary staging rack 30. In this example the staging rack 30 has locations for 24 individual capillaries in a single row. The staging rack positions the lower end of each capillary at a specified height. This insures that each capillary processed through the system will make contact with fluid in a microwell plate filled to a specific level. The staging rack also allows the capillary manipulator, under computer control, to withdraw from 1 to 12 capillaries for processing. When at least 12 capillaries have been withdrawn from the rack, the capillary manipulator then transfers a row of 12 capillaries from the capillary rack 28 and places them immediately adjacent to any remaining capillaries in staging rack 30. The staging rack is movable between two positions fore and aft under computer control so that capillaries are always withdrawn contiguously from one end of staging rack 30. This insures that when 12 or fewer capillaries remain in the staging rack there will be at least 12 contiguous positions into which a row of 12 capillaries can be transferred from capillary rack 28.

Adjacent to the staging rack 30 is an optical capillary detector 32. The optical detector contains a light source and a photocell on opposite sides of a slot in the top of the detector 32. For sufficiently large capillaries, this device may be what is commonly described in the field of electronics as a photointerrupter. Whenever it is desirable to verify that a capillary is being held in a particular position by a capillary manipulator, the capillary manipulator is moved to pass the capillary through the slot of the detector 32. If there is a capillary in the particular position of the manipulator it will interrupt the light beam between the source and the photocell. This interruption is sensed by the computer controlling the capillary manipulator which then is assured that a capillary is in the tested position of the capillary manipulator.

In accordance with the principles of the present invention the micro-volume immunoassay system 110 includes a capillary manipulator comprising a capillary gripper 140 mounted on robotic actuators 142, 144 and 146. In this example the robotic actuators 142, 144, 146 are motorized linear translation stages and are arranged to provide x, y, z motion control although other actuator mechanisms could also be employed as long as they are computer controllable. The gripper 140 can move up and down by operation of the up-down actuator 142. The actuator 142 is moved from front to back by actuator 144. Actuator 144 in turn is moved between the left and right sides of the system 110 by and in relation to actuator 146. In FIGS. 15 and 16 the gripper 140 is seen to be holding twelve capillaries in a vertical orientation in which the capillaries are generally transported and filled. However, the gripper is hinged at its connection to the actuator 142 by a hinge 148 so that it can be controllably pivoted 90°, thereby moving the capillaries to a horizontal orientation. When the gripper and capillaries are in this orientation, capillaries can be put into and removed from the capillary holders 120′ and 120″. The combined actuator mechanisms thus can traverse all of the elements of the system in front of the detection and separation and immobilization modules.

The robotic actuators 142, 144, 146 in this example manipulate four tools. In addition to the gripper 140 the robotic actuators manipulate a lid remover 154, a pipette 150 connected to a syringe pump 152 and a reformatting gripper 140a, shown in FIG. 23. When one of the three tools 154, 150, 140a is to be used, it is lowered to a position below the level of gripper 140 by a respective vertical actuator (not shown) and then into an operating position by actuators 142, 144, 146. This prevents one tool from interfering with any other tool during use. The lid remover performs tasks such as removing lids which cover the microwell plates to prevent evaporation of the fluids inside the microwells. The lid remover does this by first lowering into position over the lid to be removed, applying suction created by a vacuum source to hold the lid and then raising up to lift the lid off the microwell plate. The lids (if used) of the small bottles 158 can also be removed and replaced by the lid remover 154 in a similar manner. In an alternative implementation, the wells of the microwell plates 24 may be foil sealed prior to being placed into the system 110. When fluid access is required, the seal could be punctured with a tool carried by actuators 142, 144, 146 to expose the well(s) to be accessed. The pipette 150 performs precise fluid transport such as filling a capillary holder reservoir 124 with fluids such as electrophoretic buffers or luminol contained in small bottles 158 near the front of the baseplate 112.

The capillary reformatting gripper 140a, described more fully in FIGS. 22a-22c, performs the task of changing the spacing of capillaries between 9 mm and 2.25 mm. It is desirable to space capillaries more closely in the detection module to improve collection efficiency by creating an object area that aligns with that of the CCD detector. In the illustrated example, the reformatting gripper 40a is configured to hold three capillaries on a 9 mm center-to-center spacing corresponding to that of gripper 140, although grippers capable of holding a different number of capillaries and on different spacings could also be used. As described previously, gripper 140 can place up to 12 capillaries into holders 120′ and 120″ on a 9 mm center-to-center spacing. Gripper 140a is then used to pick up three of the capillaries (e.g., capillaries 1,2,3) from holder 120″ and place them immediately adjacent to and spaced 2.25 mm center-to-center from the other capillary positions of the capillary holder 120′ (e.g., capillary positions 1,4,7). This process is repeated three more times so that all capillaries are interlaced to a 2.25 mm center-to-center final spacing as FIG. 17e illustrates. Other spacings and configurations are also possible.

The gripper 140, which will be more fully described below, is moved under computer-control to the capillaries which are to be picked up and processed (e.g., loaded with sample) or moved to another operation in the system. The gripper 140 can be programmed to pick up one capillary at a time or a number of capillaries simultaneously, such as a row of capillaries, from the capillary staging rack 30. In this example, the gripper can then dip the lower end of the capillaries into a row of corresponding microwells simultaneously or each capillary in succession into a single microwell to completely fill each capillary in the gripper by capillary action or by vacuum applied to the upper end of the capillary. Each capillary has thus functioned as a volumetric pipette where the volume contained corresponds to the volume of the capillary lumen. By filling a number of capillaries from a single microwell maximum utilization can be made of fluid, conserving expensive reagents.

The system 110 includes a wash trough 156 which is filled either by pipette 150 from a bottle 158, or directly from syringe pump 152 and is used to wash the capillaries or the pipette tip. In the illustrated example the lower end of all twelve capillaries held by the gripper can be inserted into the wash trough at the same time. At the far end of the wash trough 156 is a small well which is separate from the main fluid compartment of the trough. This small well can be used for mixing small amounts of fluids to minimize fluid consumption during use of the system. As previously mentioned, capillaries may also be washed while positioned horizontally in a capillary holder 120′ by electrically pumping fluid contained within reservoirs 124 of the holder.

FIGS. 17a-17e illustrate various examples of a capillary holder suitable for use in the system of FIGS. 15 and 16. The capillary holder 120′ of FIG. 17a is made of a non-wetting material such as Teflon® or a rubber-like material to take advantage of fluid surface tension. On opposite ends of the capillary holder are fluid reservoirs 124. In the illustrated example these fluid reservoirs extend continuously for the length of the holder. However in a constructed implementation it is alternatively possible to subdivide the reservoirs 124 into separate compartments so that different capillaries in the holder can be isolated from one another. This would enable individual control or monitoring of each capillary as well as the ability to utilize different fluids. The central area 122 of the holder 120′ is recessed so that capillaries may be gripped and deposited and removed from the holder. The inner walls of the reservoirs 124 are formed on one side as a series of V-grooves 126 which retain capillaries. The series of V-grooves 126 are deep enough that whenever a capillary is deposited into the V-groove it will automatically drop into a position defined by a V-groove 126. The back surface of the capillary holder 120′ (not visible in this illustration) has two electrodes 134 extending from the back side of the holder, each of which protrudes through the wall of the holder and into a reservoir 124. These electrodes are used to apply a potential through the fluid in the capillaries for 25 electrophoresis and isoelectric focusing. Capillary holders may be removed from the system for cleaning if required.

FIG. 17b shows a capillary holder 120. In this example, instead of a central recessed area, the central area 132 of the holder 120 is open. This opening 132 enables the holder to be placed over a CCD detector to acquire photons emitted from substances inside the capillaries. With the example 120′ of FIG. 17a the capillaries must be opposed to a CCD detector located above the capillaries. With the example of FIG. 17b the CCD detector can approach the capillaries from either above or below. Placing the CCD above the capillary holder has the advantages of enabling visualization of the full length of capillary and eliminating the possibility of any liquid spilling onto the CCD or imaging optics. In the view of FIG. 17b the electrodes 134 extending from each reservoir 124 can be seen on the front side of the capillary holder.

FIG. 17c shows the capillary holder 120′ with eight capillaries 160 located in the V-grooves. The number of V-grooves, and hence the number of capillaries that can be held in a holder, is a matter of design choice.

FIG. 17d is a view of a cross section of a capillary holder of the preceding examples. In this example the electrodes 136 extend to and through the lateral sidewalls of the reservoirs 124. The reservoirs 124 are shown filled with a fluid 130. The fluid 130 in each reservoir is seen to be higher than the lowest point in the V-grooves 126 where the capillary 160 is supported. When the capillary is placed in the V-groove 126 it breaks through the surface tension of the fluid 130 in each reservoir, which completely immerses the aperture at each end of the capillary 160 in fluid. However, because of the non-wetting material of the holder and capillary 160, the surface tension of the fluid 130 is not disturbed to a degree that would cause the fluid to leak into the central area 122 of the capillary holder. The fluid path of the capillary 160 remains in fluidic contact with the fluid 130 in each reservoir (and therefore with the electrodes 136) without any need for a physical seal by virtue of the surface tension of the fluid 130. This is particularly advantageous since there are no moving parts to wear out and making temporary seals to multiple small capillaries can be complicated and expensive to implement. In addition, this approach is significantly more scalable. For continuous chemiluminescent detection as described above, the height of the luminol fluid in one reservoir is higher than the fluid level in the other reservoir, enabling the luminol to flow through the capillary 160 from one reservoir to the other by hydrodynamic flow.

FIG. 17e shows intermediate capillary holder 120″ with a capacity of twelve capillaries 160, along with a capillary holder 120′. The nine capillaries located in the intermediate holder 120″ may be repositioned by reformatting gripper 140a into capillary holder 120′ as previously described by picking up three capillaries at a time from holder 120″ and placing them in an interlaced sequence in the holder 120′ until the holder 120′ is filled.

In the examples described above, capillaries are held in the capillary holder V-grooves by gravity. In another implementation, capillaries may be secured in the capillary holder (e.g., mechanically or by vacuum). This may be desirable as a means to prevent static charge on the capillaries or any other surface, causing the capillaries to not rest properly in the capillary holder V-grooves. Alternatively, the capillaries may be coated with an antistatic material, or an ionizing source may be incorporated into the system to prevent static charge.

An example of a capillary gripper 140 is shown in FIGS. 18-21. The gripper of FIG. 18 is configured to hold twelve capillaries on a 9 mm center-to-center spacing, although grippers capable of holding different numbers of capillaries and with different spacings can also be configured. The gripper of FIG. 18 comprises a body 80 from which twelve fingers 82 extend. Near the distal end of each finger 82 is a groove 86 in which a capillary can be captured. The groove is normally covered by an L-shaped spring clamp 84. In their unflexed state, shown in FIG. 18, the spring clamps 84 cover the grooves 86, thereby holding capillaries in the grooves 86 of the fingers. When a gripper is to be opened to pick up or release a capillary a clamp actuator inside the body 80, described below, is actuated to flex the distal ends of the spring clamps 84 away from the fingers 82, thus exposing the grooves 86 and releasing any capillary held in a groove. In the example shown, the clamp actuator for the spring clamps actuates to flex all of the spring clamps to an open position simultaneously. Alternatively, the clamp actuator can be arranged to individually open only selected spring clamps of the gripper. After the force of the clamp actuator is released the spring force of the spring clamp(s) returns the spring clamp(s) to the closed condition. FIG. 19 illustrates the gripper of FIG. 18 when holding twelve capillaries 160. FIG. 20 is an enlarged view of the fingers 82 in which one finger 82′ is holding a capillary 160 and the end finger 82″ is shown with the spring clamp removed, revealing a slot 88 in the finger which contains a pin 192 of the clamp actuator. When the clamp actuator is actuated to open a spring clamp, the pin 192 moves out of its slot 88 toward the spring clamp of the finger, thereby pushing the spring clamp away from the distal end of the finger where the groove 86 is covered.

FIG. 21 is a rear view of the gripper body 80 which illustrates the clamp actuator 190. The fingers 82 can be seen extending from the opposite side of the body at the bottom of the drawing figure. The pins 192 in each of the fingers are connected to the clamp actuator 190. The clamp actuator 190 is coupled to an air cylinder 194. When air pressure is applied to the air cylinder, the piston of the air cylinder pushes the clamp actuator to the left, which pushes the pins 192 against the spring clamps 84 and opens the gripper fingers. When air pressure is removed the spring clamps close. In another implementation, vacuum can be used to pull the air cylinder and the attached clamp actuator to the open position. An advantage of using vacuum or negative air pressure is that the same vacuum source can provide vacuum for the lid remover as well as other pneumatic actuators within the system. By choosing the appropriate pneumatic mechanism, all four tools on the robotic actuators, the capillary gripper 140, capillary reformatting gripper 140a, the pipette 150 and the lid remover 154, can be automatically operated from a common pressure or vacuum source.

As an alternative to the air cylinder an electromagnetic solenoid can be used to move the clamp actuator 190. A single solenoid can be used to move a unitary clamp actuator connected to all of the pins 192 of the gripper, or individual solenoids can be used for each pin to permit separate operation and control of each capillary gripper finger.

FIGS. 22a and 22b illustrate a reformatting gripper 140a that is comprised of a body 1102 from which three thin fingers 1104 extend. At the distal end of each finger is a groove 1106 in which a capillary can be captured. At the bottom of each groove is a small hole 1108 that is connected through a valve to a vacuum source. When the gripper 140a is positioned such that a capillary is engaged in the groove 1106 at the end of a finger 1104 and vacuum is applied, the capillary will be captured in the groove as shown in FIG. 22b and therefore movable to a new position before release by removal of vacuum. Gripper 140a is capable of spacing capillaries more closely than possible with the design of gripper 140 by virtue of the end capture feature although capillaries are not held as securely. FIG. 23 is a side view of system 110 which shows a reformatting gripper 140a located on actuator 142.

FIGS. 24a, 24b and 24c show a vacuum manifold 1110 that may be used in conjunction with gripper 140. The manifold engages the upper end of each capillary through ports 1112 such that vacuum may be applied to the upper end of each capillary and cause fluid flow up through each capillary. The manifold 1110 may contain a vacuum chamber 1114 common to all ports or isolated vacuum ports for individual capillaries, depending on the degree of control required. The capillaries engage the vacuum ports loosely such that when vacuum is applied, some air may flow around the outside of the capillaries and into the ports at a velocity great enough to sweep away any fluid droplets formed at the capillary ends. The total flow capacity of the vacuum source is selected so that the flow around the capillaries does not adversely affect the vacuum level at the ends of the capillaries. The rate of flow may be varied by adjusting the level of vacuum. With the lowest level of vacuum, there is only enough pressure to assist filling the capillaries without causing continuous flow. At higher levels of vacuum, which may cause droplets to be formed and swept away, the applied vacuum may be adjusted to vary the rate of flow. The vacuum may also be pulsed to cause intermittent flow conditions, such as on and off or high and low, which can be important for specific processes or reduced fluid consumption.

In an alternate implementation, where electrical pumping of fluid (e.g., electrophoresis) is employed, the capillary may physically engage the manifold port (e.g., by a light friction fit through a hole in a membrane) to achieve a low pressure fluidic seal. Buffer is added to the region above the capillary such that an electrical connection is made from the capillary to an electrode integrated into the manifold. An array of electrodes in microwells or other fluid containers adjacent to the far ends of the capillaries completes the electrical path for pumping fluids from the wells of a microwell plate 28 or trough 156 through the capillaries. A computer controlled power supply provides the necessary control.

In another implementation, fluid may be pumped through the capillaries by pressure.

In yet another implementation, flow can be caused by wicking, blotting or evaporating fluid from one end of a capillary while the other end is in contact with liquid or air.

In still another implementation, fluid can be pumped through the capillaries from a reservoir 124 while they are positioned in a capillary holder 120′ (described below) by application of voltage across electrodes 136.

The system 110 is completely computer controlled and operated by a separate computer with programs and interfaces to control and operate the mechanisms of the system 110, in particular the robotic actuators 142, 144, 146, the capillary gripper 140, capillary reformattor 140a, the pipette 150, the lid remover 154, the computer-controlled power supply and UV light inside the separation and immobilization module 118, and the CCD array detector and light emitting diode array inside the detection module. In addition, the movable trays for the capillary holders 120′, 120″ and capillary staging rack 30 may also be moved under computer control.

In use, the system operator will begin by placing all reagents and capillaries into the instrument, then selecting an operating protocol containing the processing steps to be carried out. The protocol is preferably selected from a protocol list stored in the computer which operates the system and is displayed on the graphic user interface of the system. The operator will also enter parameters which define particular features such as the locations of fluids which are to be accessed, where the fluids are to be mixed, voltages for electrophoresis and isoelectric focusing and the like. The steps of the operating protocol can be precisely defined because a capillary precisely defines the amount of fluid and substances needed for a process by the internal volume of the capillary. A typical protocol may begin by pumping wash fluid from bulk bottle 114 into the trough 156. The robotic actuators 142, 144, 146 move the pipette 150 to a position above solution bottle 158, lower the pipette to immerse the tip into the solution, and the syringe pump is computer actuated to withdraw a predetermined amount of solution from the bottle. The pipette is then lifted by the robotic actuators, moved above capillary holder 120′ of the separation and immobilization module, then lowered at which point the solution is dispensed into one of the capillary holder reservoirs 124 to fill the reservoir above the bottom level of the V-grooves 126. The pipette is then moved to another bottle 158 where a predetermined amount of solution is withdrawn by the syringe pump and dispensed into the other reservoir 124 in capillary holder 120′. A typical processing solution may be an electrophoretic buffer. The pipette is then moved to the wash trough and the tip is washed.

Capillaries are taken from the storage rack 28 by the capillary gripper 140 and placed in the capillary staging rack 30. The capillaries are now at a known, uniform height for further handling by the gripper.

The system then acquires samples for analysis which the reference lab has received from a patient or referring physician. This process may begin with the robotic actuators 142, 144, 146 moving the lid remover 154 over a covered microwell plate and removing the cover. The robotic actuators then move the capillary gripper 140 to the staging rack 30 where the gripper picks up a number of capillaries 160. The capillaries are moved to a position above the uncovered microwell plate 24 and the robotic actuators lower the gripper so that the ends of the capillaries are dipped into the fluids in a number of microwells. Each microwell contains analytes such as a standard mixture of known proteins or peptides associated with the target pathology. When the end of a capillary touches an analyte solution the fluid wicks up into and fills the lumen of the capillary with the solution. The capillaries are then lifted and the lid is replaced on the microwell plate. Next, the gripper with the filled capillaries is moved up and over the capillary holders 120′ and 120″ and then pivoted so that the capillaries 160 are positioned horizontally. The gripper then is lowered to place the capillaries into the V-grooves of the capillary holders. Finally, the capillaries are re-formatted to a 2.25 mm center-to-center spacing, by the gripper 140a, into the capillary holder 120′ which is then retracted into the separation and immobilization module 118 by the movable tray 19 of the module.

When the capillary holder is moved into the separation and immobilization module 118, a computer-selected voltage is applied across the fluid path of the capillary, establishing a pH gradient in the capillary which separates and distributes the analytes inside the capillary by isoelectric focusing. Once the standard analytes have been separated they are bound at their locations in the capillary by photoactivation with UV light. Visible light, thermal, chemical activation or other means of immobilizing proteins (or other molecules, substances, etc.) may also be employed in which case an activating mechanism other than the UV light may be utilized in the separation and immobilization module. The binding may be covalent or non-covalent such as by hydrophobic or ionic interaction. After the standard analytes have been bound in place in the capillary the movable tray moves the capillary holder out of the separation and immobilization module and the re-formatting gripper 140a positions the capillaries on 9 mm centers in capillary holders 120′ and 120″ for pickup by gripper 140.

The capillary gripper 140 then removes the capillaries from the capillary holders and unbound material is washed away by first dipping the lower end of the capillaries into the wash trough 156 and then applying vacuum to the manifold at the upper end to effect fluid flow. As described previously, fluids can also be moved by electrical pumping or other means. Next, the lid is removed from a microwell plate 24 containing a patient-derived serum to be assayed, the capillary ends are dipped into the serum liquid in the wells and patient serum is flowed through the capillaries, allowing antibodies specific for the target pathology to bind to their corresponding peptides or proteins. The wash process is repeated to remove unbound antibody. As a result of dipping the capillary into the wash solution, antibody is also removed from the outside. A secondary antibody to which a detectable marker is added (e.g., goat anti-human in the case of HIV) is pumped through the capillaries from yet another set of wells, binding occurs and then the capillaries are washed to remove unbound material.

Following the wash and binding steps described above, a luminol solution is mixed in either a microwell or in the separate well of the wash trough 56. Luminol and activator are contained in bottles 158. Pipette 150 is used to transfer the solutions from bottles 58 into the mixing well. The solutions are mixed by repeatedly aspirating and dispensing the fluid in the mixing well. To minimize the amount of luminol used, only a small amount is prepared in the selected well by carefully metering the fluid with the pipette 150. Each of the capillaries are dipped into and therefore filled with the luminol solution. The remaining prepared luminol is then transferred to one of the two reservoirs 124 of a capillary holder 120′ and water is dispensed into the other reservoir 124. Since the luminol is intended to flow hydrodynamically through the capillary in order to continuously stimulate chemiluminescence from the bound antibody-target complexes, slightly more volume of luminol is injected into one reservoir than water into the other to promote the desired flow. The capillaries are then placed in the capillary holder 120′ containing the luminol by the capillary gripper 140 and re-formatting gripper 140a. The capillary holder 120′ is moved into the detection module 116. Inside the detection module the capillary holder is positioned with the capillaries in opposition to the CCD array detector, which in this example is above the capillaries. As luminol flows through the capillaries chemiluminescence is induced and the photons produced are detected by the CCD array detector in relation to the locations from which the photons are emitted, revealing the presence of pathogen-specific antibodies in the patient serum. The detected data is received by the computer and processed into a desired display on the system display which may be, for example, a graph of light intensity versus location within the capillary or bars sized and located in a row as a function of location within the capillary, much in the manner of the familiar Western blot pattern, and the results recorded by the computer. To detect fluorescently labeled molecules within the capillaries, the capillaries are irradiated by a light source and the resulting fluorescence is detected by the same CCD array. In this way the fluorescence data may be accurately overlaid spatially with the chemiluminescence data. As an alternative to a CCD array, in such an implementation a fluorescent scanner may be used.

Upon completion of detection, the capillaries are removed from the capillary holder of the detection module 116 and discarded into a capillary waste container (not shown) on baseplate 112.

One skilled in the art will appreciate that the above procedure is merely representative of one experimental protocol which may be performed by an assay system of the present invention. By editing the protocol provided by the system or entering a new protocol, the system operator has the ability to add, omit, and vary the processing steps used in a given experiment.

As previously mentioned, the computer system which controls the actuators, tools, power supply, UV light source, and CCD array of an immunoassay system of the present invention also preferably has a graphical user interface by which the system operator can select an operating run protocol, initialize the system, execute the protocol run, and store and analyze the results in a reliable, convenient and easy to operate manner. The graphical user interface has a means such as a menu, directory or listing by which the system operator can select default run protocols, protocols stored from previously executed runs or can prepare a custom protocol. The protocol provides a sequence of instructions to the computerized system as to how to manipulate the reagents in order to produce the desired results. The selected run protocol may be presented on the graphical user interface as a sequence of steps, as a flowchart or other presentation of the protocol sequence. During execution of the protocol by the automated system the graphical user interface may display the status of the current assay and, at the conclusion, display the results. Further details of this automated assay system may be found in U.S. patent application Ser. No. 11/401,699, filed Apr. 10, 2006, the contents of which are incorporated herein by reference.

An implementation of the present invention may be used to measure a patient's serum antibodies directed against disease proteins such as those of HIV, BSE, Lyme disease, and other conditions. In such an implementation of the present invention, a standard or known specimen of one or more proteins is resolved in a capillary such as by isoelectric focusing and immobilized as by photoimmobilization. The capillary now contains one or more known proteins in separate locations in the capillary. The patient sample to be assayed is applied to the capillary and patient antibodies to the proteins, when present, will bind to the immobilized proteins in the capillary. The bound antibodies are detected as by tagging the bound complexes with chemiluminescent markers. An implementation of the present invention may thus be used to measure pathogen-derived proteins, peptides, or other detectable molecules, as shown by the following examples.

Measuring Antibodies Directed Against HIV Disease Proteins:

A standard mixture of known proteins or peptides associated with the pathology, such as peptide products of HIV proteins, are loaded by the reference lab into capillaries, focused and immobilized. This can be done prior to performance of the actual assay using patient samples, and the capillaries stored until use. In this case, this step may be performed by the manufacturer of the capillaries prior to delivery of the capillaries to the reference lab. Alternatively, the capillaries can be prepared immediately prior to performance of the assay. At the time of the assay, patient-derived serum is run through the capillary, and any antibodies specific for the pathogen-related peptides or proteins bind to the peptides or proteins. After washing, a secondary antibody such as goat anti-human, to which a detectable marker has been attached, is run through the capillary to bind patient-derived antibodies bound to the capillary. Detection by chemiluminescence or fluorescence reveals the presence of pathogen-specific antibodies in the patient sample. A positive result signature would exist where multiple serum antibodies bind multiple protein or peptide targets arrayed within the capillary by earlier isoelectric focusing.

Measuring Antibodies Directed Against BSE Disease Proteins:

In the case of an assay for BSE, a brain tissue sample is collected from the brain stem of the animal suspected of being infected with BSE prion protein. This sample is homogenized in an appropriate buffer. The homogenate is then treated with proteinase K digestion enzyme. The resistance to proteinase K of the prion form of the protein allows the prion protein to persist, whereas the non-prion form of the protein is cleaved by proteinase K. The products of this treatment are then mixed with an appropriate separation buffer, introduced to a capillary, and resolved by isoelectric focusing. After focusing, an ultraviolet light activates photochemistry, binding resolved proteins and peptides to the capillary. After immobilization, the capillary is washed by flowing buffer through it. BSE-prion-specific primary antibody is then flowed into the capillary allowing it to bind to the BSE-prion-specific protein. A second washing step removes unbound antibody. A secondary antibody coupled to a reporter enzyme is then flowed into the capillary allowing it to bind to the primary antibody. Finally, chemiluminescent substrate is flowed through the capillary causing the generation of light in the regions of the capillary where the prion specific protein was immobilized and primary and secondary antibodies attached to it. The emitted light is detected by a CCD camera. Following this procedure the BSE-prion protein gives a characteristic banding pattern that identifies an infected animal. This method may also be used for other prior-type diseases including scrapie and Creutzfeldt-Jakob disease.

Measuring Antibodies Directed Against Lyme Disease Proteins:

In the case of an assay for Lyme disease, cells of Borrelia burgdorferi, the causative agent of Lyme disease, are used to prepare a cell lysate in a lysis buffer. This lysate is then mixed with an appropriate buffer system for carrying out isoelectric focusing (IEF). Capillaries are filled with this lysate/IEF buffer mixture, and placed between catholyte and anolyte buffer chambers. An electrical potential is then supplied causing proteins in the capillary to be resolved by IEF. After focusing, an ultraviolet light source is applied, exposing the capillaries to U.V. light, thereby activating photochemistry that causes resolved proteins to bind to the wall of the capillaries. Once bound within the capillaries, unbound materials are washed from these by flowing an appropriate buffer solution through the capillaries. This is followed by filling the capillaries with blood serum of patients suspected of having Lyme disease. If Lyme disease is present, the patient's antibodies against Borrelia burgdorferi proteins will bind to the Borrelia burgdorferi proteins that have been focused into bands and immobilized within the capillaries. After allowing patient antibodies to bind their targets, a second wash step removes unbound patient antibodies. A secondary antibody containing a detectable marker and that binds to human antibodies is then flowed into the capillary. This secondary antibody binds to patient-derived antibodies that in turn are bound to target Borrelia burgdorferi proteins bound to the capillaries. Finally, chemiluminescent substrate is flowed through the capillaries causing the generation of light in the regions of the capillaries where the Borrelia burgdorferi proteins were immobilized and patient primary antibody and labeled secondary antibodies were attached to them. The emitted light is detected by a CCD camera. Following this procedure the Borrelia burgdorferi proteins give a characteristic banding pattern that identifies a patient sample as coming from an infected patient with antibodies against these proteins.

Measuring Pathogen-Derived Proteins, Peptides, or Other Detectable Molecules:

Patient-derived material such as plasma or tissue samples are analyzed in this application. If cellular tissue is used, this assay typically begins with a cell lysis step. Isoelectric focusing and photochemical capture are used to resolve sample content and immobilize them within the capillary, as described above. Pathology-specific antibodies are then applied to the capillary and allowed to bind to their targets. After washing, application of secondary antibodies, and detection substrate, a pattern of detected pathogen-specific analytes within the capillary is detected. In some specific cases, such as prion protein detection, a proteolytic digestion step may precede analysis, analogous to the assay now performed commercially for detection of prion disease using Western blotting.

Measuring Antibodies Directed Against an Analyte:

From the forgoing, it is also apparent that a broadly applicable method for measuring antibodies against an analyte can be defined. A standard mixture of analytes (e.g., 1 of FIG. 3A) is loaded in a capillary (FIG. 3B), focused (FIG. 3D) and immobilized (FIG. 3E). Such analytes may include but are not limited to whole cells or viral particles, solubilized components of whole cells or viral particles, purified naturally occurring proteins, heterologously expressed proteins, synthetic peptides, and other synthetic compounds. This focusing and immobilization can be done prior to performance of the actual assay for measuring antibodies, and the capillaries stored until use. In this case, this step may be performed by the manufacturer of the capillaries prior to delivery of the capillaries to the user. Alternatively, the capillaries can be prepared immediately prior to performance of the assay. At the time of the assay, serum is run through the capillary, and any antibodies specific for the analyte bind to the analyte. After washing, in the case of human serum being analyzed, a secondary antibody such as goat anti-human, to which a detectable marker has been attached (FIG. 3F), is run through the capillary to bind patient-derived antibodies bound to the capillary (FIG. 3G). Similarly for non-human serum, a secondary antibody with attached detectable marker and that which binds to the antibodies of the species from which the serum was obtained may be used. Detection by chemiluminescence, fluorescence or other detection means reveals the presence of analyte-specific antibodies in the serum sample (FIG. 3H). A positive result signature would exist where multiple serum antibodies bind multiple analyte targets arrayed within the capillary by earlier isoelectric focusing.

What is claimed is:

1. A method for performing a protein assay comprising:
loading a biological sample of one or more known analytes into a capillary;
resolving analytes of the sample in the capillary;
immobilizing the resolved analytes in the capillary;
binding patient antibodies to the immobilized analytes; and
detecting the location of antibody-analyte complexes.

2. The method of claim 1, wherein the patient antibodies are contained in patient serum and wherein loading is preceded by performing a screening test on the patient serum.

3. The method of claim 2, wherein performing a screening test comprises performing an ELISA assay.

4. The method of claim 2, wherein detecting further comprises detecting the location of patient antibodies which are particular to HIV.

5. The method of claim 4, wherein detecting further comprises detecting the locations of a plurality of patient antibodies which are particular to a plurality of proteins associated with HIV.

6. The method of claim 2, wherein detecting further comprises detecting the location of patient antibodies which are particular to Lyme disease.

7. The method of claim 1, wherein resolving comprises resolving analytes in the capillary by isoelectric focusing.

8. The method of claim 7, wherein detecting is preceded by flowing a chemiluminescent substrate through the capillary.

9. The method of claim 1 wherein the steps of resolving, immobilizing, binding and detecting are performed by an integrated automated computer-controlled assay system.

10. The method of claim 9, wherein the integrated automated computer-controlled assay system further performs the step of loading.

11. An automated assay system for detecting the presence of antibodies to a disease condition comprising:
a processing station at which one or more known resolved proteins are immobilized in a capillary;
a capillary gripper which is operable to manipulate capillaries at the processing station; and
a detection station, wherein the capillary gripper is further operable to manipulate processed capillaries containing a patient sample at the detection station,
wherein the presence of patient antibodies to one or more of the known proteins is detected at the detection station.

12. The automated assay system of claim 11, wherein the capillary gripper further comprises an automated, computer-controlled capillary gripper.

13. The automated assay system of claim 12, wherein the capillary gripper is operable to mechanically grip a capillary.

14. The automated assay system of claim 12, wherein the capillary gripper is operable to mechanically grip a capillary by a vacuum.

15. The automated assay system of claim 11, wherein the capillary gripper is further operable to load a biological sample into a capillary at the processing station.

16. The automated assay system of claim 11, further comprising a microtiter plate located at the processing station.

17. The automated assay system of claim 11, further comprising a photon detector located at the detection station.

18. The automated assay system of claim 11 further comprising a user interface for configuring a protocol by which the automated assay system is to analyze the biological sample.

19. The automated assay system of claim 18, wherein the user interface is also operable to display detection results.

20. A method for performing an assay comprising:
resolving pathogen-derived analytes in a capillary;
immobilizing the resolved analytes in the capillary;
loading patient-derived serum into the capillary;
allowing binding of patient-serum-derived antibodies to the immobilized analytes; and
detecting the location of serum-derived antibodies by means of a detection agent.

21. The method of claim 20, wherein resolving is preceded by performing a screening test on the patient-derived serum.

22. The method of claim 21, wherein performing a screening test comprises performing an ELISA assay.

23. The method of claim 21, wherein detecting further comprises detecting the location of analytes which are particular to HIV.

24. The method of claim 21, wherein detecting further comprises detecting the location of analytes which are particular to Lyme disease.

25. The method of claim 20, wherein resolving comprises resolving analytes in the capillary by isoelectric focusing.

26. The method of claim 25, wherein allowing binding comprises allowing antibodies to bind to the immobilized analytes.

27. The method of claim 26, wherein detecting is preceded by flowing a chemiluminescent substrate through the capillary.

28. The method of claim 20 wherein the steps of resolving, immobilizing, loading and detecting are performed by an integrated automated computer-controlled assay system.

29. The method of claim 20, wherein resolving and immobilizing are performed prior to the time that a patient-derived serum is assayed,
whereby the patient-derived serum assay begins with a capillary pre-loaded with pre-resolved and pre-immobilized analytes.

30. A method for performing an assay comprising:
resolving analytes in a capillary;
immobilizing the resolved analytes in the capillary;
loading serum into the capillary;
allowing serum-derived antibodies to bind to the immobilized analytes; and
detecting the location of serum-derived antibodies by means of a detection agent.

31. The method of claim 30, wherein the serum comprises human biological material.

32. The method of claim 30, wherein the serum comprises non-human biological material.

33. The method of claim 30, further comprising introducing a secondary antibody to which a detectable marker has been attached prior to detecting.

34. The method of claim 33, wherein allowing serum-derived antibodies to bind produces serum-derived antibody-analyte complexes; and
wherein the secondary antibody comprises an antibody which binds to the serum-derived antibody-analyte complexes.

35. The method of claim 33, further comprising washing prior to introducing a secondary antibody.

36. The method of claim 30, wherein resolving analytes further comprises resolving one or more known analytes in a capillary.

37. The method of claim 36, wherein resolving one or more known analytes further comprises resolving at least one of whole cells, viral particles, purified naturally occurring proteins, heterologously expressed proteins, or synthetic peptides.

38. The method of claim 30, wherein detecting further comprises detecting by one of chemiluminescence or fluorescence detection.

39. The method of claim 30, wherein resolving comprises resolving a plurality of analytes of a standard in a capillary;
wherein immobilizing comprises immobilizing the plurality of analytes at different locations in the capillary;
wherein loading serum comprises loading serum including a plurality of antibodies to the plurality of analytes into the capillary; and
wherein detecting comprises detecting multiple locations of serum-derived antibodies.

40. The method of claim 30 wherein the steps of resolving, immobilizing, allowing serum-derived antibodies to bind, and detecting are performed by an integrated automated computer-controlled assay system.

41. The method of claim 30, further comprising:
recording results from the detection.

42. The method of claim 41 wherein the steps of loading, allowing to bind, detecting and recording are performed by an integrated automated computer-controlled assay system.
